SEQUENCE LISTING (15 sequences)

SEQ ID NO: 1 - DNA, 51 bp, Homo sapiens
  misc_feature (26)..(26): can be C or T
ctcttcactatgttgaaattgggtcnttcttccccaaagattgaagagaat

SEQ ID NO: 2 - DNA, 51 bp, Homo sapiens
  misc_feature (26)..(26): can be A or G
tcagatgggaaatattgcaggggctntatggtctccatcgcaactactcac

SEQ ID NO: 3 - DNA, 51 bp, Homo sapiens
  misc_feature (26)..(26): can be A or C
ttttgagactggcaaatgttctgcanccagtatctgctcaatacttttgtg

SEQ ID NO: 4 - DNA, 51 bp, Homo sapiens
  misc_feature (26)..(26): can be C or G
caaaagtctttaggatagtctctggntcacagtaagtgctacgtaagtgtt

SEQ ID NO: 5 - DNA, 51 bp, Homo sapiens
  misc_feature (26)..(26): can be A, G or T
tttttatacatgttattttagggcanaagctgagtactatacccccacacc

SEQ ID NO: 6 - DNA, 51 bp, Homo sapiens
  misc_feature (26)..(26): can be C or G
gaggtaggagaatcacttgaacccangggtcagaggttgtggtgagccgag

SEQ ID NO: 7 - DNA, 61 bp, Homo sapiens
agtttgcaaagtaacccatttggccatgcagactgtaagtcatacaactctagagggacaa

SEQ ID NO: 8 - DNA, 51 bp, Homo sapiens
  misc_feature (26)..(26): can be C or T
ctcctatctccattttactcttatgnctacccccagaataggttttctgga

SEQ ID NO: 9 - DNA, 51 bp, Homo sapiens
  misc_feature (26)..(26): can be absent or T
ggtggtaatgtatatttatcttaaantttttttttttttttgagacggagt

SEQ ID NO: 10 - DNA, 51 bp, Homo sapiens
  misc_feature (26)..(26): can be A or G
gcggatcgcctgagatcaggagttcnagaccagcctggccaacatggtgaa

SEQ ID NO: 11 - DNA, 51 bp, Homo sapiens
  misc_feature (26)..(26): can be A or G
cgcaatggtgtgatctcagctcactncaacctctaactcccaggttcaagc

SEQ ID NO: 12 - DNA, 51 bp, Homo sapiens
  misc_feature (26)..(26): can be C or T
ctgcctacaaaagtatcaggcaaganaggcctcacgttagatgagatagta

SEQ ID NO: 13 - DNA, 51 bp, Homo sapiens
  misc_feature (26)..(26): can be absent or A
ggcaataagagtgaaactccatctcnaaaaaaaaaaaaaaaaaatctattt

SEQ ID NO: 14 - DNA, 50 bp, Homo sapiens
agtttgcaaagtaacccatttggccaagtcatacaactctagagggacaa

SEQ ID NO: 15 - DNA, 51 bp, Homo sapiens
  misc_feature (26)..(26): n can be A or G
acctcctcccataaattgcagaatcnattcccttcctgcccactctcagtg

(19) United States
(12) Patent Application Publication
(10) Pub. No.: US 2022/0259656 A1
(43) Pub. Date: Aug. 18, 2022

(54) BLOOD BIOMARKER AND GENETIC MARKERS ASSOCIATED WITH RESPONSE TO CRTH2 RECEPTOR ANTAGONISTS

(71) Applicants: Devan V. Mehrotra, North Wales, PA (US); George Philip, North Wales, PA (US); Peter M. Shaw, West Point, PA (US); Merck Sharp & Dohme Corp., Rahway, NJ (US)

(72) Inventors: Devan V. Mehrotra, Lansdale, PA (US); George Philip, Gwynedd Valley, PA (US); Peter M. Shaw, Yardley, PA (US)

(73) Assignee: Merck Sharp & Dohme Corp., Rahway, NJ (US)

(21) Appl. No.: 17/606,232

(22) PCT Filed: Apr. 28, 2020
(86) PCT No.: PCT/US2020/030176

§ 371 Date: Oct. 25, 2021

Related U.S. Application Data
(60) Provisional application No. 62/842,990, filed on May 3, 2019.

(51) Int. Cl. C12Q 1/6883; C07K 14/705

(52) U.S. Cl. CPC C12Q 1/6883; C12Q 2600/106; C07K 14/705

(57) ABSTRACT

The present invention provides a blood biomarker and genetic markers on human chromosome 1 that are associated with a beneficial response to CRTH2 receptor antagonists. These combination of the blood biomarker and genetic markers are useful, inter alia, to identify patients who are most likely to benefit from treatment with CRTH2 receptor antagonist compositions and drug products, in methods of treating patients having a disease susceptible to treatment with a CRTH2 receptor antagonist, e.g., asthma, and in methods for selecting the most appropriate therapy for such patients.

### FIELD OF THE INVENTION

The present invention relates to a blood biomarker and genetic markers on human chromosome 1 that are predictive of a beneficial response to therapy with CRTH2 receptor antagonists.

### BACKGROUND OF THE INVENTION

Identification of any publication in this section or any section of this application is not an admission that such publication is prior art to the present invention.

Asthma is a highly prevalent disease associated with significant morbidity and mortality, and accounting for high direct and indirect healthcare expenditures. World Health Organization (WHO) data currently estimate the prevalence of asthma to be 300 million individuals worldwide, with this number expected to increase to 400 million by 2025. It is estimated that approximately 15 million disability adjusted life years are lost to asthma, and one in 250 deaths is due to asthma. Masoli M, et al., for the Global Initiative for Asthma (GINA) Program, Allergy 2004;59:469-78. This high disease burden is in part due to patients who are not well controlled on standard therapy. Bateman E D, et al., Am J Respir Crit Care Med 2004;170:836-44. In addition, compliance with standard inhaler therapy is relatively low. It is estimated that 44.2% and 51.5% of patients who begin a combination and concurrent inhalational therapy, respectively, do not renew their initial prescription during the first year. Marceau C, et al., Allergy Clin Immunol 2006;118:574-81. Alternative options to inhalers include oral agents, such as montelukast and zileuton, as well as methylxanthines such as aminophylline; however, these agents are recognized to be less potent than inhaled agents. Therefore, a need exists for new, well-tolerated oral therapies that effectively treat asthma, either alone or in combination with available therapies.

Chemoattractant Receptor-homologous molecule on Th2 cells (CRTH2) is a G protein-coupled receptor for the prostaglandin D2 (PGD2) expressed on eosisnophils, basophils and Th2 cells. In vitro, PGD2 and some of its CRTH2-selective metabolites can recruit and activate these leukocytes by 1) stimulating the expression of the surface protein CD11b which favors cell adhesion to the vascular wall and transmigration of cells from the blood circulation to the inflamed tissue and 2) stimulating cell movement to the site of inflamation (chemotaxis). CRTH2 activation also leads to the stimulation of Th2 cytokines release, such as IL-13 from the TH2 cells and to the stimulation of basophil and eosinophil degranulation.

Existing pre-clinical and clinical data suggest that the PGD2/CRTH2 pathway is fundamental to the recruitment and activation of key pro-inflammatory leukocytes contributing to asthma. Shichijo M, Arimura A, Hirano Y, et al. Clin Exp Allergy 2009 Sep;39(9):1404-14; Lukacs N W, Berlin A A, Franz-Bacon K, et al. Am J Physiol Lunch Cell Mol Physiol 2008:295:L767-79; Uller L, Mathiesen J M, Alenmyr L, et al. Respir Res 2007;8:161. In humans, a CRTH2 genetic polymorphism leading to increased CRTH2 mRNA stability is significantly associated with asthma in two independent populations. Huang J-L, Gao P-S, Mathias R A, Yao T-C, Chen L-C, Kuo M-L, et al. Hum Mol Genet 2004;13(21):2691-7. Ramatroban, a dual TP/CRTH2 antagonist is reported to exhibit some degree of efficacy in allergic rhinitis and is commercialized in Japan. Ishizuka T, Matsui T, Okamoto Y, et al., Cardiovasc Drug Rev 2004;22:71-90. Furthermore a recent Phase IIa clinical study conducted in a patient population afflicted with eosinophilic severe asthma, demonstrated reduction of sputum eosinophils in patients treated with of the CRTH2 antagonist, fevipiprant. Gonem S, Berair R, Singapuri A, et al. Lancet Respir Med. 2016 (9):699-707. Taken together, these findings are consistent with a potentially important role for CRTH2 inhibition in the treatment of asthma.

U.S. Patent Application Publication No. US20180237856 discloses genetic markers on human chromosome 1 that are associated with a beneficial response to CRTH2 receptor antagonists. In some embodiments, the publication discloses a method of treating a patient with a disorder susceptible to treatment with a CRTH2 receptor antagonist, e.g., asthma, comprising administering a therapeutically effective amount of the CRTH2 receptor antagonist to the patient, wherein the patient, prior to the administration of the CRTH2 receptor antagonist has tested positive for at least one copy of a better response allele selected from a specified list of CRTH2 receptor antagonist markers.

The therapeutic effect of CRTH2 receptor antagonists can vary widely among patients afflicted with asthma. In order to better target patients who might respond better to CRTH2 receptor and thereby provide a better and more cost-effective treatments for asthma, a need exists for methods of identifying patients who are most likely to benefit through treatments with CRTH2 receptor antagonists. This invention addresses that need.

### SUMMARY OF THE INVENTION

The present invention is based on the discovery that the presence of a particular blood marker and specific genetic polymorphisms such as single nucleotide polymorphisms (SNP) on human chromosome 1 are significantly associated with response to treatment with CRTH2 receptor antagonists in patients suffering from a disorder associated with CRTH2 receptor function. The specific genetic polymorphisms associated with response to CRTH2 receptor antagonist therapy are referred to herein as the “Genetic Markers.” The present invention may guide medical practitioners, health authorities, and medical insurance providers in selecting a suitable population of asthmatic patients which might benefit from CRTH2 receptor antagonist therapy.

One of the criteria in identifying patients who respond to treatment is the determination of a blood biomarker which is, namely, the determination of a blood eosinophil count of at least 250 eosinophils/microliter. In a study population of asthmatic patients, described below in Example 1, subjects having a blood eosinophil count of ≥300 eosinophils/microliter and who also tested positive for the presence of at least one better response allele of a particular genetic polymorphism were associated with about a 3-fold better improvement in Forced Expiratory Volume in one second (FEV₁) compared to those having a blood eosinophil count of <300 eosinophils/microliter .

The Genetic Marker criteria used in study in Example 1 was a SNP, which has a C/T polymorphism, identified as rs12748961 in the NCBI SNP Database. The presence of a C allele (i.e., a C/C or C/T genotype) in combination with a blood eosinophil count ≥300 eosinophils/microliter was associated with the better treatment response in the study. While the C allele is the minor allele of the rs12748961 in Caucasians, it is present at a substantially higher frequency in a population of Asian ancestry than in the overall population. Other genetic polymorphisms which can also serve as the genetic criteria in identifying patients associated with a beneficial response to a CRTH2 receptor antagonist, e.g., improvements in FEV₁ score in asthmatic patients, are polymorphisms on chromosome 1. The genetic polymorphisms associated with a beneficial response to CRTH2 receptor antagonist therapy are described in Table 1 below, wherein PS means polymorphic site according to the SNP NCBI database and “-” in the second column indicates that the variant represents a deletion or insertion variant. As an example of a deletion variant, the alternate allele in rs67625805 represents a deletion of the T nucleotide at the corresponding position. In another example, rs71970505, an 11-residue nucleotide segment is absent in one of the alleles, where as in the alternative allele, the nucleotide segment ATGCAGACTGT is present at the corresponding position of the nucleotide sequence.

The genetic polymorphisms other than rs12748961 in Table 1 were identified by genetic imputation as described in Example 2 of U.S. Patent Application Publication No. US20180237856. In addition any genetic variants in high Linkage disequilibrium with SNPs in Table 1 can also serve as beneficial response markers

TABLE 1
Genetic Markers

| | | Better |
|---|---|---|
| Polymorphic | | Response |
| Site (PS) | Alleles | Allele |
| rs12748961 | T/C | C |
| rs12118655 | A/G | G |
| rs6679073 | C/A | A |
| rs12564209 | C/G | G |
| rs3805 | T/G/A | G |
| rs71633561 | G/C | C |
| rs71970505 | ATGCAGACTGT/— | — |
| rs12132270 | C/T | T |
| rs67625805 | T/— | — |
| rs3747972 | A/G* | A |
| rs11557080 | G/A | A |
| rs71633563 | C/T | T |
| rs34848415 | A/— | — |
| rs1891091 | A/G* | A |

*The NCBI database indicates that rs3747972 and rs1891091 are RefSNP Alleles for the reverse strands.

The inventors herein contemplate that testing individuals for the presence of a high blood eosinophil count and at least one or more of the Genetic Markers in Table 1 or a linked variant of one or more of the Genetic Markers will be useful in a variety of pharmacogenetic products and methods that involve identifying subjects most likely to respond to therapy for CRTH2 receptor antagonists for disorders susceptible to treatment with CRTH2 receptor antagonists. The method of the present invention will also assist physicians in deciding whether to prescribe a CRTH2 receptor antagonist to a patient afflicted with asthma. For instance, the inventors contemplate that testing subjects for the presence of a blood eosinophil count at least 250 eosinophils/microliter and for the presence of at least one copy of the C allele for the rs12748961 SNP will be useful for such products and methods, and in assisting physicians.

Accordingly, in embodiment no. 1, the invention provides a method of treating a patient with a disorder susceptible to treatment with a CRTH2 receptor antagonist comprising:
administering a therapeutically effective amount of the CRTH2 receptor antagonist to the patient,
wherein said patient, prior to the administration of the CRTH2 receptor antagonist, has:
   (a) a blood eosinophil count of at least 250 eosinophils/microliter; and
   (b) tested positive for at least one copy of a better response allele selected from a Genetic Marker in Table 1 above or a linked variant of one or more of the Genetic Markers.

In a first aspect of embodiment no. 1, said patient, prior to the administration of the CRTH2 receptor antagonist, has tested positive for at least one copy of the C allele of the rs12748961 SNP.

In a second aspect of embodiment no. 1, said patient, prior to the administration of the CRTH2 receptor antagonist, has tested positive for at least one copy of the G allele of the rs12118655 SNP.

In a third aspect of embodiment no. 1, said patient, prior to the administration of the CRTH2 receptor antagonist, has tested positive for at least one copy of the A allele of the rs6679073 SNP.

In a fourth aspect of embodiment no. 1, said patient, prior to the administration of the CRTH2 receptor antagonist, has tested positive for at least one copy of the G allele of the rs12564209 SNP.

In a fifth aspect of embodiment no. 1, said patient, prior to the administration of the CRTH2 receptor antagonist, has tested positive for at least one copy of the G allele of the rs3805 SNP.

In a sixth aspect of embodiment no. 1, said patient, prior to the administration of the CRTH2 receptor antagonist, has tested positive for at least one copy of the C allele of the rs71633561 SNP.

In a seventh aspect of embodiment no. 1, said patient, prior to the administration of the CRTH2 receptor antagonist, has tested positive for at least one copy of the deletion allele of the rs71970505 SNP (indicated as “-” in the Better Response Allele column of Table 1).

In an eighth aspect of embodiment no. 1, said patient, prior to the administration of the CRTH2 receptor antagonist, has tested positive for at least one copy of the T allele of the rs12132270 SNP.

In a ninth aspect of embodiment no. 1, said patient, prior to the administration of the CRTH2 receptor antagonist, has tested positive for at least one copy of the deletion allele of the rs67625805 SNP (indicated as “-” in the Better Response Allele column of Table 1).

In a tenth aspect of embodiment no. 1, said patient, prior to the administration of the CRTH2 receptor antagonist, has tested positive for at least one copy of the A allele of the rs3747972 SNP.

In an eleventh aspect of embodiment no. 1, said patient, prior to the administration of the CRTH2 receptor antagonist, has tested positive for at least one copy of the A allele of the rs11557080 SNP.

In a twelfth aspect of embodiment no. 1, said patient, prior to the administration of the CRTH2 receptor antagonist, has tested positive for at least one copy of the T allele of the rs71633563 SNP.

In a thirteenth aspect of embodiment no. 1, said patient, prior to the administration of the CRTH2 receptor antagonist, has tested positive for at least one copy of the deletion allele of the rs34848415 SNP (indicated as “-” in the Better Response Allele column of Table 1).

In a fourteenth aspect of embodiment no. 1, said patient, prior to the administration of the CRTH2 receptor antagonist, has tested positive for at least one copy of the A allele of the rs1891091 SNP.

In a fifteenth aspect of embodiment no. 1 (including any one of the first-fourteenth aspects), the patient has a blood eosinophil count of at least 300 eosinophils/microliter.

In a sixteenth aspect of embodiment no. 1 (including any one of the first-fifteenth aspects), the method further comprises administering a leukotriene antagonist such as montelukast, zafilukast, or pranlukast to the patient. In a seventeeth aspect of embodiment no. 1 (including any one of the first-sixteenth aspects), the method further comprises administering montelukast to the patient.

In embodiment no. 2, the invention provides a drug product which comprises a pharmaceutical composition and prescribing information,
wherein the pharmaceutical composition comprises a CRTH2 receptor antagonist and the prescribing information comprises a blood eosinophil count and a pharmacogenetic indication,
wherein the prescribing information comprises the treatment of a disease susceptible to treatment with the CRTH2 receptor antagonist in patients who have a blood eosinophil count of at least 250 eosinophils/microliter and who also have pharmacogenetic indication, wherein said patients also test positive for at least one copy of a better response allele selected from a Genetic Marker in Table 1 above or a linked variant of one or more of the Genetic Markers.

In a first aspect of embodiment no. 2, the pharmacogenetic indication comprises the treatment of a disease susceptible to treatment with the CRTH2 receptor antagonist in patients who test positive for at least one copy of the C allele of the rs12748961 SNP.

In a second aspect of embodiment no. 2, the pharmacogenetic indication comprises the treatment of a disease susceptible to treatment with the CRTH2 receptor antagonist in patients who test positive for at least one copy of the G allele of the rs12118655 SNP.

In a third aspect of embodiment no. 2, the pharmacogenetic indication comprises the treatment of a disease susceptible to treatment with the CRTH2 receptor antagonist in patients who test positive for at least one copy of the A allele of the rs6679073 SNP.

In a fourth aspect of embodiment no. 2, the pharmacogenetic indication comprises the treatment of a disease susceptible to treatment with the CRTH2 receptor antagonist in patients who test positive for at least one copy of the G allele of the rs12564209 SNP.

In a fifth aspect of embodiment no. 2, the pharmacogenetic indication comprises the treatment of a disease susceptible to treatment with the CRTH2 receptor antagonist in patients who test positive for at least one copy of the G allele of the rs3805 SNP.

In a sixth aspect of embodiment no. 2, the pharmacogenetic indication comprises the treatment of a disease susceptible to treatment with the CRTH2 receptor antagonist in patients who test positive for at least one copy of the C allele of the rs71633561 SNP.

In a seventh aspect of embodiment no. 2, the pharmacogenetic indication comprises the treatment of a disease susceptible to treatment with the CRTH2 receptor antagonist in patients who test positive for at least one copy of the deletion allele of the rs71970505 SNP.

In an eighth aspect of embodiment no. 2, the pharmacogenetic indication comprises the treatment of a disease susceptible to treatment with the CRTH2 receptor antagonist in patients who test positive for at least one copy of the T allele of the rs12132270 SNP.

In a ninth aspect of embodiment no. 2, the pharmacogenetic indication comprises the treatment of a disease susceptible to treatment with the CRTH2 receptor antagonist in patients who test positive for at least one copy of the deletion allele of the rs67625805 SNP.

In a tenth aspect of embodiment no. 2, the pharmacogenetic indication comprises the treatment of a disease susceptible to treatment with the CRTH2 receptor antagonist in patients who test positive for at least one copy of the A allele of the rs3747972 SNP.

In an eleventh aspect of embodiment no. 2, the pharmacogenetic indication comprises the treatment of a disease susceptible to treatment with the CRTH2 receptor antagonist in patients who test positive for at least one copy of the A allele of the rs11557080 SNP.

In a twelfth aspect of embodiment no. 2, the pharmacogenetic indication comprises the treatment of a disease susceptible to treatment with the CRTH2 receptor antagonist in patients who test positive for at least one copy of the T allele of the rs71633563 SNP.

In a thirteenth aspect of embodiment no. 2, the pharmacogenetic indication comprises the treatment of a disease susceptible to treatment with the CRTH2 receptor antagonist in patients who test positive for at least one copy of the deletion allele of the rs34848415 SNP.

In a fourteenth aspect of embodiment no. 2, the pharmacogenetic indication comprises the treatment of a disease susceptible to treatment with the CRTH2 receptor antagonist in patients who test positive for at least one copy of the A allele of the rs1891091 SNP.

In a fifteenth aspect of embodiment no. 2 (including any one of the first-fourteenth aspects), the patient has a blood eosinophil count of at least 300 eosinophils/microliter.

In a sixteenth aspect of the drug product set forth in embodiment no. 2 (including any one of the first-fifteenth aspects), the drug product further comprises a leukotriene antagonist such as montelukast, zafilukast, or pranlukast. In a seventeenth aspect of embodiment no. 2 (including any one of the first-fifteenth aspects) the drug product further comprises montelukast.

In embodiment no.3, the invention provides the use of a CRTH2 receptor antagonist in the manufacture of a medicament for treating a patient having:
a disease susceptible to treatment with a CRTH2 receptor antagonist,
a blood eosinophil count of at least 250 eosinophils/microliter, and
a positive test for at least one copy of the better response allele selected from a Genetic Marker as set forth in Table 1 above or a linked variant of one or more of the Genetic Markers.

In a first aspect of embodiment no. 3, the patient has a positive test for at least one copy of the C allele of the rs12748961 SNP.

In a second aspect of embodiment no. 3, the patient has a positive test for at least one copy of the G allele of the rs12118655 SNP.

In a third aspect of embodiment no. 3, the patient has a positive test for at least one copy of the A allele of the rs6679073 SNP.

In a fourth aspect of embodiment no. 3, the patient has a positive test for at least one copy of the G allele of the rs12564209 SNP.

In a fifth aspect of embodiment no. 3, the patient has a positive test for at least one copy of the G allele of the rs3805 SNP.

In a sixth aspect of embodiment no. 3, the patient has a positive test for at least one copy of the C allele of the rs71633561 SNP.

In a seventh aspect of embodiment no. 3, the patient has a positive test for at least one copy of the deletion allele of the rs71970505 SNP.

In an eighth aspect of embodiment no. 3, the patient has a positive test for at least one copy of the T allele of the rs12132270 SNP.

In a ninth aspect of embodiment no. 3, the patient has a positive test for at least one copy of the deletion allele of the rs67625805 SNP.

In a tenth aspect of embodiment no. 3, the patient has a positive test for at least one copy of the A allele of the rs3747972 SNP.

In an eleventh aspect of embodiment no. 3, the patient has a positive test for least one copy of the A allele of the rs11557080 SNP.

In a twelfth aspect of embodiment no. 3, the patient has a positive test for least one copy of the T allele of the rs71633563 SNP.

In a thirteenth aspect of embodiment no. 3, the patient has a positive test for at least one copy of the deletion allele of the rs34848415 SNP.

In a fourteenth aspect of embodiment no. 3, the patient has a positive test for at least one copy of the A allele of the rs1891091 SNP.

In a fifteenth aspect of embodiment no. 3 (including any one of the first-fourteenth aspects), the patient has a blood eosinophil count of at least 300 eosinophils/microliter.

In embodiment no. 4, the invention provides a method of selecting a therapy for treating a patient having a disease susceptible to treatment with a CRTH2 receptor antagonist, in which a patient's blood eosinophil count and genotype at a polymorphic site selected from those set forth in Table 1 above or a linked variant of one or more of the Genetic Markers are determined and reported, the method comprising:
consulting the report to identify that the patient has a blood eosinophil count of at least 250 eosinophils/microliter and at least one copy of the better response allele of the Genetic Marker; and
based on that consultation, treating the patient with the CRTH2 receptor antagonist.

In a first aspect of embodiment no. 4, the report is consulted to identify that the patient has at least one copy of the C allele of the rs12748961 SNP.

In a second aspect of embodiment no. 4, the report is consulted to identify that the patient has at least one copy of the G allele of the rs12118655 SNP.

In a third aspect of embodiment no. 4, the report is consulted to identify that the patient has at least one copy of the A allele of the rs6679073 SNP.

In a fourth aspect of embodiment no. 4, the report is consulted to identify that the patient has at least one copy of the G allele of the rs12564209 SNP.

In a fifth aspect of embodiment no. 4, the report is consulted to identify that the patient has at least one copy of the G allele of the rs3805 SNP.

In a sixth aspect of embodiment no. 4, the report is consulted to identify that the patient has at least one copy of the C allele of the rs71633561 SNP.

In a seventh aspect of embodiment no. 4, the report is consulted to identify that the patient has at least one copy of the deletion allele of the rs71970505 SNP.

In an eighth aspect of embodiment no. 4, the report is consulted to identify that the patient has at least one copy of the T allele of the rs12132270 SNP.

In a ninth aspect of embodiment no. 4, the report is consulted to identify that the patient has at least one copy of the deletion allele of the rs67625805 SNP.

In a tenth aspect of embodiment no. 4, the report is consulted to identify that the patient has at least one copy of the A allele of the rs3747972 SNP.

In an eleventh aspect of embodiment no. 4, the report is consulted to identify that the patient has at least one copy of the A allele of the rs11557080 SNP.

In a twelfth aspect of embodiment no. 4, the report is consulted to identify that the patient has at least one copy of the T allele of the rs71633563 SNP.

In a thirteenth aspect of embodiment no. 4, the report is consulted to identify that the patient has at least one copy of the deletion allele of the rs34848415 SNP.

In a fourteenth aspect of embodiment no. 4, the report is consulted to identify that the patient has at least one of the A allele of the rs1891091 SNP.

In a fifteenth aspect of embodiment no. 4 (including any one of the first-fourteenth aspects), the patient has a blood eosinophil count of at least 300 eosinophils/microliter.

In embodiment no. 5, the invention provides a screening method for selecting patients for treatment with a CRTH2 receptor antagonist from a group of patients having a disorder susceptible to treatment with the CRTH2 receptor antagonist, comprising testing each member of the group for a blood eosinophil count of at least 250 eosinophils/microliter and for the presence of at least one copy of the better response allele of a Genetic Marker selected from those set forth in Table 1 above or a linked variant of one or more of the Genetic Markers, wherein a positive test is the finding of a blood eosinophil count of at least 250 eosinophils/microliter and the presence of at least one copy of the better response allele of the Genetic Marker or a linked variant thereof.

In a first aspect of embodiment no. 5, each member of the group is tested for the presence of at least one copy of the C allele of the rs12748961 SNP, wherein a positive test is the finding of a blood eosinophil count of at least 250 eosinophils/microliter and the presence of at least one copy of the C allele of the rs12748961 SNP.

In a second aspect of embodiment no. 5, each member of the group is tested for the presence of at least one copy of the G allele of the rs12118655 SNP, wherein a positive test is the finding of a blood eosinophil count of at least 250 eosinophils/microliter and the presence of at least one copy of the G allele of the rs12118655 SNP.

In a third aspect of embodiment no. 5, each member of the group is tested for the presence at least one copy of the A allele of the rs6679073 SNP, wherein a positive test is the finding of a blood eosinophil count of at least 250 eosinophils/microliter and the presence of at least one copy of the A allele of the rs6679073 SNP.

In a fourth aspect of embodiment no. 5, each member of the group is tested for the presence at least one copy of the G allele of the rs12564209 SNP, wherein a positive test is the finding of a blood eosinophil count of at least 250 eosinophils/microliter and the presence of at least one copy of the G allele of the rs12564209 SNP.

In a fifth aspect of embodiment no. 5, each member of the group is tested for the presence at least one copy of the G allele of the rs3805 SNP, wherein a positive test is the finding of a blood eosinophil count of at least 250 eosinophils/microliter and the presence of at least one copy of the G allele of the rs3805 SNP.

In a sixth aspect of embodiment no. 5, each member of the group is tested for the presence at least one copy of the C allele of the rs71633561 SNP, wherein a positive test is the finding of a blood eosinophil count of at least 250 eosinophils/microliter and the presence of at least copy of the C allele of the rs71633561 SNP.

In a seventh aspect of embodiment no. 5, each member of the group is tested for the presence at least one copy of the deletion allele of the rs71970505 SNP, wherein a positive test is the finding of a blood eosinophil count of at least 250 eosinophils/microliter and the presence of at least one copy of the deletion allele of the rs71970505 SNP.

In an eighth aspect of embodiment no. 5, each member of the group is tested for the presence at least one copy of the T allele of the rs12132270 SNP, wherein a positive test is the finding of a blood eosinophil count of at least 250 eosinophils/microliter and the presence of at least one copy of the T allele of the rs12132270 SNP.

In a ninth aspect of embodiment no. 5, each member of the group is tested for the presence at least one copy of the deletion allele of the rs67625805 SNP, wherein a positive test is the finding of a blood eosinophil count of at least 250 eosinophils/microliter and the presence of at least one copy of the deletion allele of the rs67625805 SNP.

In a tenth aspect of embodiment no. 5, each member of the group is tested for the presence at least one copy of the A allele of the rs3747972 SNP, wherein a positive test is the finding of a blood eosinophil count of at least 250 eosinophils/microliter and the presence of at least one copy of the A allele of the rs3747972 SNP.

In an eleventh aspect of embodiment no. 5, each member of the group is tested for the presence at least one copy of the A allele of the rs11557080 SNP, wherein a positive test is the finding of a blood eosinophil count of at least 250 eosinophils/microliter and the presence of at least one copy of the A allele of the rs11557080 SNP.

In a twelfth aspect of embodiment no. 5, each member of the group is tested for the presence at least one copy of the T allele of the rs71633563 SNP, wherein a positive test is the finding of a blood eosinophil count of at least 250 eosinophils/microliter and the presence of at least one copy of the T allele of the rs71633563 SNP.

In a thirteenth aspect of embodiment no. 5, each member of the group is tested for the presence at least one copy of the deletion allele of the rs34848415 SNP, wherein a positive test is the finding of a blood eosinophil count of at least 250 eosinophils/microliter and the presence of at least one copy of the deletion allele of the rs34848415 SNP.

In a fourteenth aspect of embodiment no. 5, each member of the group is tested for the presence at least one copy of the A allele of the rs1891091 SNP, wherein a positive test is the finding of a blood eosinophil count of at least 250 eosinophils/microliter and the presence of at least one copy of the A allele of the rs1891091 SNP.

In a fifteenth aspect of embodiment no. 5 (including any one of the first-fourteenth aspects), the method comprises testing each member for a blood eosinophil count of at least 300 eosinophils/microliter.

In embodiment no. 6, the invention provides a method of diagnosing a patient who is susceptible to treatment with a CRTH2 receptor antagonist and treating asthma, said method comprising:
(a) obtaining a blood sample from the patient and determining whether the patient's blood eosinophil count is at least 250 eosinophils/microliter;
(b) obtaining a biological sample from the patient and detecting whether a better response allele of at least one of the Genetic Markers in Table 1 above is present in the biological sample;
(c) diagnosing the patient as susceptible to treatment with a CRTH2 receptor antagonist when the blood eosinophil count is determined to be at least 250 eosinophils/microliter and the presence of the better response allele in the biological sample is detected; and
(d) administering a therapeutically effective amount of a CRTH2 receptor antagonist to the diagnosed patient.

In one aspect of embodiment no. 6, wherein step (d) further comprises administering a leukotriene receptor antagonist to the patient. For example, the leukotriene receptor antagonist can be montelukast or a pharmaceutically acceptable salt thereof

In one aspect of embodiment no. 6, in step (b), the C allele of the CRTH2 receptor antagonist response marker is rs12748961 SNP is detected, and
in step (c), the patient is diagnosed as susceptible to treatment with a CRTH2 receptor antagonist when the blood eosinophil count is determined to be at least 250 eosinophils/microliter and the C allele of the rs12748961 SNP is detected.

In another aspect of embodiment no. 6, in step (b), the G allele of the CRTH2 receptor antagonist response marker is rs12118655 SNP is detected, and
in step (c), the patient is diagnosed as susceptible to treatment with a CRTH2 receptor antagonist when the blood eosinophil count is determined to be at least 250 eosinophils/microliter and the G allele of the rs12118655 SNP is detected.

In another aspect of embodiment no. 6, in step (b), the A allele of the CRTH2 receptor antagonist response marker is rs6679073 SNP is detected, and
in step (c), the patient is diagnosed as susceptible to treatment with a CRTH2 receptor antagonist when the blood eosinophil count is determined to be at least 250 eosinophils/microliter and the A allele of the rs6679073 SNP is detected.

In another aspect of embodiment no. 6, in step (b), the G allele of the CRTH2 receptor antagonist response marker is rs12564209 SNP is detected, and
in step (c), the patient is diagnosed as susceptible to treatment with a CRTH2 receptor antagonist when the blood eosinophil count is determined to be at least 250 eosinophils/microliter and the G allele of the rs12564209 SNP is detected.

In another aspect of embodiment no. 6, in step (b), the G allele of the CRTH2 receptor antagonist response marker is rs3805 SNP is detected, and
in step (c), the patient is diagnosed as susceptible to treatment with a CRTH2 receptor antagonist when the blood eosinophil count is determined to be at least 250 eosinophils/microliter and the G allele of the rs3805 SNP is detected.

In another aspect of embodiment no. 6, in step (b), the C allele of the CRTH2 receptor antagonist response marker is rs71633561 SNP is detected, and
in step (c), the patient is diagnosed as susceptible to treatment with a CRTH2 receptor antagonist when the blood eosinophil count is determined to be at least 250 eosinophils/microliter and the C allele of the rs71633561 SNP is detected.

In another aspect of embodiment no. 6, in step (b), the deletion allele of the CRTH2 receptor antagonist response marker is rs71970505 SNP is detected, and
in step (c), the patient is diagnosed as susceptible to treatment with a CRTH2 receptor antagonist when the blood eosinophil count is determined to be at least 250 eosinophils/microliter and the deletion allele of the rs71970505 SNP is detected.

In another aspect of embodiment no. 6, in step (b), the T allele of the CRTH2 receptor antagonist response marker is rs12132270 SNP is detected, and
in step (c), the patient is diagnosed as susceptible to treatment with a CRTH2 receptor antagonist when the blood eosinophil count is determined to be at least 250 eosinophils/microliter and the T allele of the rs12132270 SNP is detected.

In another aspect of embodiment no. 6, in step (b), the deletion allele of the CRTH2 receptor antagonist response marker is rs67625805 SNP is detected, and
in step (c), the patient is diagnosed as susceptible to treatment with a CRTH2 receptor antagonist when the blood eosinophil count is determined to be at least 250 eosinophils/microliter and the deletion allele of the rs67625805 SNP is detected.

In another aspect of embodiment no. 6, in step (b), the A allele of the CRTH2 receptor antagonist response marker is rs3747972 SNP is detected, and
in step (c), the patient is diagnosed as susceptible to treatment with a CRTH2 receptor antagonist when the blood eosinophil count is determined to be at least 250 eosinophils/microliter and the A allele of the rs3747972 SNP is detected.

In another aspect of embodiment no. 6, in step (b), the A allele of the CRTH2 receptor antagonist response marker is rs11557080 SNP is detected, and
in step (c), the patient is diagnosed as susceptible to treatment with a CRTH2 receptor antagonist when the blood eosinophil count is determined to be at least 250 eosinophils/microliter and the A allele of the rs11557080 SNP is detected.

In another aspect of embodiment no. 6, in step (b), the T allele of the CRTH2 receptor antagonist response marker is rs71633563 SNP is detected, and
in step (c), the patient is diagnosed as susceptible to treatment with a CRTH2 receptor antagonist when the blood eosinophil count is determined to be at least 250 eosinophils/microliter and the T allele of the rs71633563 SNP is detected.

In another aspect of embodiment no. 6, in step (b), the deletion allele of the CRTH2 receptor antagonist response marker is rs34848415 SNP is detected, and
in step (c), the patient is diagnosed as susceptible to treatment with a CRTH2 receptor antagonist when the blood eosinophil count is determined to be at least 250 eosinophils/microliter and the deletion allele of the rs34848415 SNP is detected.

In another aspect of embodiment no. 6, in step (b), the A allele of the CRTH2 receptor antagonist response marker is rs1891091 SNP is detected, and
in step (c), the patient is diagnosed as susceptible to treatment with a CRTH2 receptor antagonist when the blood eosinophil count is determined to be at least 250 eosinophils/microliter and the A allele of the rs34848415 SNP is detected.

In one aspect of embodiment no. 6, in step (d) the diagnosed patient is administered an effective amount of the CRTH2 receptor antagonist {(7R)-4-fluoro-7-[5-(4-fluorobenzyl)-1H-[1,2,3]triazol-1-yl]-6,7,8,9-tetrahydropyrido[1,2-a]indol-10-yl}-acetic acid and the leukotriene receptor antagonist montelukast.

In one particular aspect of embodiment no. 6,
in step (b), the better response allele that is sought to be detected is the C allele of the CRTH2 receptor antagonist response marker is rs12748961 SNP;
in step (c), the patient is diagnosed as susceptible to treatment with a CRTH2 receptor antagonist when the blood eosinophil count is determined to be at least 250 eosinophils/microliter and the C allele of the rs12748961 SNP is detected; and
in step (d), the diagnosed patient is administered an effective amount of the CRTH2 receptor antagonist {(7R)-4-fluoro-7-[5-(4-fluorobenzyl)-1H-[1,2,3]triazol-1-yl]-6,7,8,9-tetrahydropyrido[1,2-a]indol-10-yl}-acetic acid and the leukotriene receptor antagonist montelukast.

In embodiment no. 7, the invention provides for the (i) method of embodiment no. 1, (ii) drug product of embodiment no. 2, (iii) use of embodiment no. 3, (iv) method of embodiment no. 4, (v) method of embodiment no. 5, and (vi) the method of embodiment no. 6, wherein the patient is susceptible to treatment with a CRTH2 receptor antagonist has been determined to have of a blood eosinophil count of at least 250 eosinophils/microliter and a positive test for at least one copy of the better response allele for at least two of the Genetic Markers in Table 1 above. For example, in this embodiment, the patient susceptible to treatment with a CRTH2 receptor antagonist is identified where patients have both at least one copy of the C allele of the rs12748961 SNP and at least one copy of the G allele of the rs12118655 SNP (as well as a finding of a blood eosinophil count of at least 250 eosinophils/microliter).

In certain embodiments of the methods, uses, or drug products, described in the embodiments above, the CRTH2 receptor antagonist is {(7R)-4-fluoro-7-[5-(4-fluorobenzyl)-1H-[1,2,3]triazol-1-yl]-6,7,8,9-tetrahydropyrido[1,2-a]indol-10-yl}-acetic acid or 2-(2-methyl-1-(4-(methylsulfonyl)-2-(trifluoromethyl)benzyl)-1H-pyrrolo[2,3-b]pyridin-3-yl)acetic acid (fevipiprant), or a pharmaceutically acceptable salt of either compound.

In certain embodiments of the methods, uses, drug products, or kits described in the embodiments above, the disorder susceptible to treatment with a CRTH2 receptor antagonist is asthma.

### BRIEF DESCRIPTION OF THE DRAWINGS

FIG. 1 shows reference nucleotide sequences for the Genetic Markers with the variant position indicated in bold font in the NCBI SNP database as of June 7, 2015.

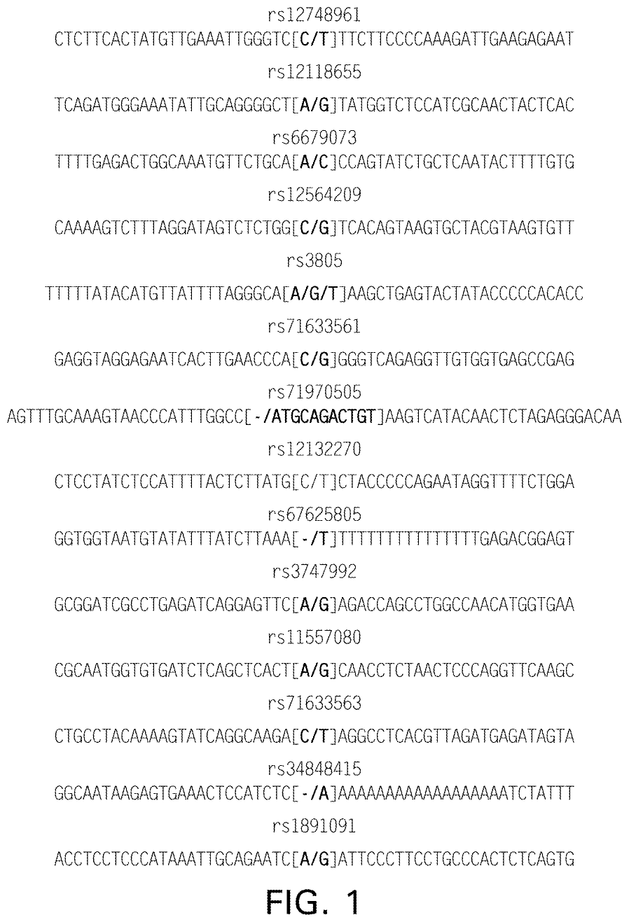

FIG. 2 is graphical depiction of a study design used in measuring the efficacy of patients who tested positive for the presence of at least one C allele of the rs 12748961 SNP and were treated with a CRTH2 receptor antagonist and montelukast.

### DETAILED DESCRIPTION OF THE INVENTION

### I. Definitions.

So that the invention may be more readily understood, certain technical and scientific terms are specifically defined below. Unless specifically defined elsewhere in this document, all other technical and scientific terms used herein have the meaning that would be commonly understood by one of ordinary skill in the art to which this invention belongs when used in similar contexts as used herein.

As used herein, including the appended claims, the singular forms of words such as “a,” “an,” and “the,” include their corresponding plural references unless the context clearly dictates otherwise.

“About” when used to modify a numerically defined parameter, e.g., the dosage for a therapeutic agent discussed herein, or the length of treatment time, means that the parameter may vary by as much as 10% above or below the stated numerical value for that parameter.

“Allele” is a particular form of a gene or other genetic locus, distinguished from other forms by its particular nucleotide sequence, the term allele also includes one of the alternative polymorphisms (e.g., a SNP) found at a polymorphic site.

“Beneficial result” means a desired clinical result of treatment with a CRTH2 receptor antagonist, including but not limited to: alleviation of one or more disease symptoms, diminishment of extent of disease (e.g., improvement in FEVᵢ in the context of the treatment of asthma), stabilized (i.e., not worsening) state of disease, slowing of disease progression, amelioration or palliation of a disease state, prolonging survival (as compared to expected survival if not treated), relapse-free survival, remission (whether partial or total) and cure (i.e., elimination of the disease).

“Better response allele” is the particular form of a gene or other genetic locus, where if present in a patient, results in an improved clinical measure (e.g., an improved FEV₁ measure) as compared to the measure in a patient where such form of the gene or other genetic locus is absent.

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic or novel properties of the specified dosage regimen, method, or composition.

“High blood eosinophil count” is meant to mean a blood eosinophil count of at least 250 eosinophils/microliter, preferably at least 300 eosinophils/microliter.

“Individual” or “animal” or “patient” or “mammal,” is meant any human subject, particularly a mammalian subject, for whom any of the claimed compositions and methods is needed or may be beneficial. In preferred embodiments, the individual is an adult human, i.e., at least 18 years of age.

“Isolated” is typically used to reflect the purification status of a biological molecule such as RNA, DNA, oligonucleotide, or protein, and in such context means the molecule is substantially free of other biological molecules such as nucleic acids, proteins, lipids, carbohydrates, or other material such as cellular debris and growth media. Generally, the term “isolated” is not intended to refer to a complete absence of other biological molecules or material or to an absence of water, buffers, or salts, unless they are present in amounts that substantially interfere with the methods of the present invention.

“Locus” refers to a location on a chromosome or DNA molecule corresponding to a gene, a physical feature such as a polymorphic site, or a location associated with a phenotypic feature.

“Nucleotide pair” is the set of two nucleotides (which may be the same or different) found at a polymorphic site on the two copies of a chromosome from an individual.

“Oligonucleotide” refers to a nucleic acid that is usually between 5 and 100 contiguous bases in length, and most frequently between 10-50, 10-40, 10-30, 10-25, 10-20, 15-50, 15-40, 15-30, 15-25, 15-20, 20-50, 20-40, 20-30 or 20-25 contiguous bases in length. The sequence of an oligonucleotide can be designed to specifically hybridize to any of the allelic forms of a locus;

such oligonucleotides are referred to as allele-specific probes. If the locus is a PS comprising a SNP, the complementary allele for that SNP can occur at any position within an allele-specific probe. Other oligonucleotides useful in practicing the invention specifically hybridize to a target region adjacent to a PS with their 3′ terminus located one to less than or equal to about 10 nucleotides from the PS, preferably about 5 nucleotides. Such oligonucleotides hybridizing adjacent to a PS are useful in polymerase-mediated primer extension methods and are referred to herein as “primer-extension oligonucleotides”. In a preferred embodiment, the 3′-terminus of a primer-extension oligonucleotide is a deoxynucleotide complementary to the nucleotide located immediately adjacent to the PS.

“Pharmaceutically acceptable” refers to molecular entities and compositions that are “generally regarded as safe”—e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

“Polymorphic site” or “PS” refers to the position in a genetic locus or gene at which a polymorphism is found, e.g., single nucleotide polymorphism (SNP), restriction fragment length polymorphism (RFLP), variable number of tandem repeat (VNTR), dinucleotide repeat, trinucleotide repeat, tetranucleotide repeat, simple sequence repeat, insertion element such as Alu, and deletion or insertion of one or more nucleotides. A PS is usually preceded by and followed by highly conserved sequences in the population of interest and thus the location of a PS is typically made in reference to a consensus nucleic acid sequence of thirty to sixty nucleotides that bracket the PS, which in the case of a SNP is commonly referred to as the “SNP context sequence”. The location of the PS may also be identified by its location in a consensus or reference sequence. The skilled artisan understands that the location of a particular PS may not occur at precisely the same position in a reference or context sequence in each individual in a population of interest due to the presence of one or more insertions or deletions in that individual as compared to the consensus or reference sequence. Moreover, it is routine for the skilled artisan to design robust, specific and accurate assays for detecting the alternative alleles at a polymorphic site in any given individual, when the skilled artisan is provided with the identity of the alternative alleles at the PS to be detected and one or both of a reference sequence or context sequence in which the PS occurs. Thus, the skilled artisan will understand that specifying the location of any PS described herein by reference to a particular position in a reference or context sequence is merely for convenience and that any specifically enumerated nucleotide position literally includes whatever nucleotide position the same PS is actually located at in the same locus in any individual being tested for the presence or absence of a genetic marker of the invention using any of the genotyping methods described herein or other genotyping methods well-known in the art.

“Reference SNP” or “rs” number refers to an accession number assigned to an individual SNP by the National Center for Biotechnology Information (NCBI).

“Treat” or “Treating” means to administer a therapeutic agent, such as a composition containing CRTH2 receptor antagonists described herein, internally or externally to an individual in need of the therapeutic agent. Individuals in need of the agent include individuals who have been diagnosed as having, or at risk of developing, a condition or disorder susceptible to treatment with the agent, as well as individuals who have, or are at risk of developing, one or more adverse effects of treatment with a first therapeutic agent that are susceptible to alleviation with a second therapeutic agent. Typically, the therapeutic agent is administered in a therapeutically effective amount, which means an amount effective to produce one or more beneficial results. The therapeutically effective amount of a particular agent may vary according to factors such as the disease state, age, and weight of the patient being treated, and the sensitivity of the patient, e.g., ability to respond, to the therapeutic agent. Whether a beneficial or clinical result has been achieved can be assessed by any clinical measurement typically used by physicians or other skilled healthcare providers to assess the presence, severity or progression status of the targeted disease, symptom or adverse effect. Typically, a therapeutically effective amount of an agent will result in an improvement in the relevant clinical measurement(s) over the baseline status, or over the expected status if not treated, of at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%. For instance, in one embodiment wherein the condition or disorder is asthma, a clinical measure of improvement is an improvement in the FEVᵢ measure. While an embodiment of the present invention (e.g., a treatment method or article of manufacture) may not achieve the desired clinical benefit or result in every patient, it should do so in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

### II. Utility of the Blood Biomarker and Genetic Markers

The phenotypic effect of the blood biomarker and genetic markers described herein supports the use of these markers in a variety of commercial applications, including but not limited to, clinical trials of investigational or previously approved CRTH2 receptor antagonist drugs in patients selected on the basis of the presence or absence of these markers, pharmaceutical compositions and drug products comprising a CRTH2 receptor antagonist for treating patients who have these markers, diagnostic methods, and treatment methods, which involve tailoring a patient's drug therapy based on whether the patient has these markers.

The utility of any of the commercial applications claimed herein does not require that the correlation between the presence of a blood biomarker and Genetic Markers described herein and the occurrence of the desired response to the CRTH2 receptor antagonist be observed in 100% of the individuals that receive the CRTH2 receptor antagonist; nor does it require a diagnostic method or kit to have a specific degree of specificity or sensitivity in determining the presence or absence of the blood biomarker and Genetic Marker in every individual, nor does it require that a diagnostic method claimed herein be 100% accurate in predicting for every individual whether the individual is likely to have a beneficial response to a CRTH2 receptor antagonist. Thus, the inventors herein intend that the terms “determine”, “determining” and “predicting” should not be interpreted as requiring a definite or certain result; instead these terms should be construed as meaning that a claimed method provides an accurate result for the majority of individuals, or that the result or prediction for any given individual is more likely to be correct than incorrect.

Preferably, the accuracy of the result provided by a diagnostic method of the invention is one that a skilled artisan or regulatory authority would consider suitable for the particular application in which the method is used. Similarly, the utility of the claimed drug products and treatment methods does not require that they produce the claimed or desired effect in every individual; all that is required is that a clinical practitioner, when applying his or her professional judgment consistent with all applicable norms, decides that the chance of achieving the claimed effect of treating a given individual according to the claimed method or with the claimed drug product is sufficiently high to warrant prescribing the treatment or drug product.

A. Testing for Blood Biomarker

The determination of the presence of the blood biomarker in the methods of the invention can be performed in a number of ways well-known to skilled medical practitioners. Counting eosinophils is a routine test that can be performed in a laboratory when ordered by a physician. A medical practitioner can perform an absolute eosinophil count, which is a blood test that measures the number of eosinophils. The measurement of the eosinophils can be expressed in cells/microliter of blood. Typically, blood is drawn from a vein on the inside of the elbow or the back of the hand. Typically, the testing is performed in an automated setting. In other embodiments, the testing can be performed manually, where drawn blood is placed on a microscope slide. The practitioner can stain the sample to highlight the eosinophils and other blood cells, and they will then count the number present under a microscope to give a result. Counting can be performed manually using a hemocytometer or by use of an automated analyzer.

B. Testing for a CRTH2 Antagonist Response Marker of the Invention

The presence or absence of the Genetic Markers may be detected by any of a variety of genotyping techniques commonly used in the art. Typically, such genotyping techniques employ one or more oligonucleotides that are complementary to a region containing, or adjacent to, the PS of interest. The sequence of an oligonucleotide used for genotyping a particular PS of interest is typically designed based on a context sequence for the PS.

The location, in a particular individual, of the PS identified above is in a reference coding or genomic DNA sequence surrounding the PS of interest or in one of the context sequences described in Table 2 below, or their complementary sequences.

TABLE 2
Context sequences for SNPs associated with
CRTH2 Receptor Antagonist Response

| | Short Context | SEQ |
|---|---|---|
| PS | Sequences¹ | ID NO: |
| rs12748961 | CTCTTCACTATGTTG | 1 |
| | AAATTGGGTC [symbol] TTC | |
| | TTCCCCAAAGATTGA | |
| | AGAGAAT | |
| rs12118655 | TCAGATGGGAAATAT | 2 |
| | TGCAGGGGCT [symbol] TAT | |
| | GGTCTCCATCGCAAC | |
| | TACTCAC | |
| rs6679073 | TTTTGAGACTGGCAA | 3 |
| | ATGTTCTGCA [symbol] CCA | |
| | GTATCTGCTCAATAC | |
| | TTTTGTG | |
| rs12564209 | CAAAAGTCTTTAGGA | 4 |
| | TAGTCTCTGG [symbol] TCA | |
| | CAGTAAGTGCTACGT | |
| | AAGTGTT | |
| rs3805 | TTTTTATACATGTTA | 5 |
| | TTTTAGGGCA [symbol] AAG | |
| | CTGAGTACTATACCC | |
| | CCACACC | |
| rs71633561 | GAGGTAGGAGAATCA | 6 |
| | CTTGAACCCA [symbol] GGG | |
| | TCAGAGGTTGTGGTG | |
| | AGCCGAG | |
| rs71970505 | AGTTTGCAAAGTAAC | 7 and 14 |
| | CCATTTGGCC [symbol] AAG | (-allele) |
| | TCATACAACTCTAGA | |
| | GGGACAA | |
| rs12132270 | CTCCTATCTCCATTT | 8 |
| | TACTCTTATG [symbol] CTA | |
| | CCCCCAGAATAGGTT | |
| | TTCTGGA | |
| rs67625805 | GGTGGTAATGTATAT | 9 |
| | TTATCTTAAA [symbol] TTT | |
| | TTTTTTTTTTTTGAG | |
| | ACGGAGT | |
| rs3747972 | GCGGATCGCCTGAGA | 10 |
| | TCAGGAGTTC [symbol] AG | |
| | ACCAGCCTGGCCAAC | |
| | ATGGTGAA | |
| rs11557080 | CGCAATGGTGTGATC | 11 |
| | TCAGCTCACT [symbol] CA | |
| | ACCTCTAACTCCCAG | |
| | GTTCAAGC | |
| rs71633563 | CTGCCTACAAAAGTA | 12 |
| | TCAGGCAAGA [symbol] AG | |
| | GCCTCACGTTAGATG | |
| | AGATAGTA | |
| rs34848415 | GGCAATAAGAGTGAA | 13 |
| | ACTCCATCTC [symbol] AA | |
| | AAAAAAAAAAAAAAA | |
| | ATCTATTT | |
| rs1891091 | ACCTCCTCCCATAAA | 15 |
| | TTGCAGAATC [symbol] AT | |
| | TCCCTTCCTGCCCAC | |
| | TCTCAGTG | |

¹Context sequence reported in NCBI SNP Database on Jun. 23, 2016; [symbol] indicates C or T [symbol] indicates A or G; [symbol] indicates A or C; [symbol] indicates C or G; [symbol] indicates A/G/T; [symbol] indicates C or G; [symbol] indicates the absence (-) or presence of ATGCAGACTGT; indicates C or T; [symbol] indicates the absence (-) or presence of T; [symbol] indicates A or G; [symbol] indicates A or G; [symbol] indicates C or T; [symbol] indicates the absence (-) or presence of A, and [symbol] indicates A or G.

As recognized by the skilled artisan, nucleic acid samples containing a particular PS may be complementary double stranded molecules and thus reference to a particular site on the sense strand refers as well to the corresponding site on the complementary antisense strand. Similarly, reference to a particular genotype obtained for a PS on both copies of one strand of a chromosome is equivalent to the complementary genotype obtained for the same PS on both copies of the other strand. By way of example, a C/C genotype for the rs12748961 PS on the coding strand for the gene is equivalent to a G/G genotype for that PS on the noncoding strand.

The context sequences recited herein, as well as their complementary sequence, may be used to design probes and primers for genotyping the Genetic Markers in a nucleic acid sample obtained from a human subject of interest using any of a variety of methods well known in the art that permits the determination of whether the individual has at least one copy for the better response allele. Nucleic acid molecules utilized in such methods generally include RNA, genomic DNA, or cDNA derived from RNA.

Typically, genotyping methods involve assaying a nucleic acid sample prepared from a biological sample obtained from the individual to determine the identity of a nucleotide or nucleotide pair present at one or more polymorphic sites of interest. Nucleic acid samples may be prepared from virtually any biological sample. For example, convenient samples include whole blood serum, semen, saliva, tears, fecal matter, urine, sweat, buccal matter, skin and hair. Somatic cells are preferred since they allow the determination of the identity of both alleles present at the PS of interest.

Nucleic acid samples may be prepared for analysis using any technique known to those skilled in the art. Preferably, such techniques result in the isolation of genomic DNA sufficiently pure for determining the genotype for the desired polymorphic site(s) in the nucleic acid molecule. To enhance the sensitivity and specificity of that determination, it is frequently desirable to amplify from the nucleic acid sample a target region containing the PS to be genotyped. Nucleic acid isolation and amplification techniques may be found, for example, in Sambrook, et al., Molecular Cloning: A Laboratory Manual (Cold Spring Harbor Laboratory, New York) (2001).

Any amplification technique known to those of skill in the art may be used in practicing the present invention including, but not limited to, polymerase chain reaction (PCR) techniques. PCR may be carried out using materials and methods known to those of skill in the art (See generally PCR Technology: Principals and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Matilla et al., Nucleic Acids Res. 19: 4967 (1991); Eckert et al., PCR Methods and Applications 1: 17 (1991); PCR (eds. McPherson et al., IRL Press, Oxford); and U.S. Pat. No.4,683,202. Other suitable amplification methods include the ligase chain reaction (LCR) (see Wu and Wallace, Genomics 4: 560 (1989) and Landegren et al., Science 241: 1077 (1988)), transcription amplification (Kwoh et al., Proc. Natl. Acad. Sci. USA 86: 1173 (1989)), self-sustained sequence replication (Guatelli et al., Proc. Nat. Acad. Sci. USA, 87: 1874 (1990)); isothermal methods (Walker et al., Proc. Natl. Acad. Sci. USA 89:392-6 (1992)); and nucleic acid-based sequence amplification (NASBA).

The amplified target region is assayed to determine the identity of at least one of the alleles present at a PS in the target region. If both alleles of a locus are represented in the amplified target, it will be readily appreciated by the skilled artisan that only one allele will be detected at a PS in individuals who are homozygous at that PS, while two different alleles will be detected if the individual is heterozygous for that PS.

The identity of the allele may be identified directly, known as positive-type identification, or by inference, referred to as negative-type identification. For example, where a SNP is known to be guanine or cytosine in a reference population, a PS may be positively determined to be either guanine or cytosine for an individual homozygous at that site, or both guanine and cytosine, if the individual is heterozygous at that site. Alternatively, the PS may be negatively determined to be not guanine (and thus cytosine/cytosine) or not cytosine (and thus guanine/guanine). In either case, where it is determined that at least one copy of a better response allele is present as set forth in Table 1, that determination is deemed to be a positive test result for the better response allele in the methods, uses, drug products, or kits described herein.

Identifying the allele or pair of alleles (e.g., the two nucleotides in case of a SNP) at a PS in a nucleic acid sample obtained from an individual may be accomplished using any technique known to those of skill in the art. Preferred techniques permit rapid, accurate assaying of multiple PS with a minimum of sample handling. Some examples of suitable techniques include, but are not limited to, direct DNA sequencing of the amplified target region, capillary electrophoresis, hybridization of allele-specific probes, single-strand conformation polymorphism analysis, denaturing gradient gel electrophoresis, temperature gradient electrophoresis, mismatch detection; nucleic acid arrays, primer specific extension, protein detection, and other techniques well known in the art. See, for example, Sambrook, et al., Molecular Cloning: A Laboratory Manual (Cold Spring Harbor Laboratory, New York) (2001); Ausubel, et al., Current Protocols in Molecular Biology (John Wiley and Sons, New York) (1997); Orita et al., Proc. Nat. Acad. Sci. USA 86, 2766-2770 (1989); Humphries et al., in MOLECULAR DIAGNOSIS OF GENETIC DISEASES, Elles, ed., pp. 32 1-340, 1996; Wartell et al., Nucl. Acids Res. 18:2699-706 (1990); Hsu et al. (1994) Carcinogenesis 15:1657-1662; Sheffield et al., Proc. Natl. Acad. Sci. USA 86:232-6 (1989); Winter et al., Proc. Natl. Acad. Sci. USA 82:7575 (1985); Myers et al. (1985) Nature 313:495; Rosenbaum and Reissner (1987) Biophys Chem. 265:12753; Modrich, Ann. Rev. Genet. 25:229-53 (1991); U.S. Pat. Nos. 6,300,063; 5,837,832; 5,459,039; and HuSNP Mapping Assay, reagent kit and user manual, Affymetrix Part No. 90094 (Affymetrix, Santa Clara, Calif.).

In preferred embodiments, the identity of the allele(s) at a PS is determined using a polymerase-mediated primer extension method. Several such methods have been described in the patent and scientific literature and include the “Genetic Bit Analysis” method (WO 92/15712) and the ligase/polymerase mediated genetic bit analysis (U.S. Pat. No. 5,679,524. Related methods are disclosed in WO 91/02087, WO 90/09455, WO 95/17676, and U.S. Pat. Nos. 5,302,509 and 5,945,283. Extended primers containing the complement of the polymorphism may be detected by mass spectrometry as described in U.S. Pat. No. 5,605,798.

Another primer extension method employs allele specific PCR (Ruano, G. et al., Nucl. Acids Res. 17:8392 (1989); Ruano, G. et al., Nucl. Acids Res. 19:6877-82 (1991); WO 93/22456; Turki et al., J. Gun. Invest. 95:1635-41 (1995)).

Yet another primer extension method for identifying and analyzing polymorphisms utilizes single-base extension (SBE) of a fluorescently-labeled primer coupled with fluorescence resonance energy transfer (FRET) between the label of the added base and the label of the primer. Typically, the method, such as that described by Chen et al., Proc. Nat. Acad. Sci. 94:10756-61 (1997) uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

A preferred genotyping assay is a TaqMan® SNP Genotyping Assay from Thermo Fisher Scientific, Waltham, Mass., USA, or an assay having about the same reliability, accuracy and specificity. In certain embodiments of such an assay, two allele-specific probes are used to target a specific PS, with each probe having a distinct fluorescent label bonded to it as well as a quencher molecule. In addition, two allele-specific primers are used. Upon extension of the DNA strand, the Taq DNA polymerase cleaves the fluorescent label which cleavage results in fluorescence emissions which can be detected.

In all of the above methods, the accuracy and specificity of an assay designed to detect the identity of the allele(s) at any PS is typically validated by performing the assay on DNA samples in which the identity of the allele(s) at that PS is known. Preferably, a sample representing each possible allele is included in the validation process. For diploid loci such as those on autosomal chromosomes, the validation samples will typically include a sample that is homozygous for the major allele at the PS, a sample that is homozygous for the minor allele at the PS, and a sample that is heterozygous at that PS. These validation samples are typically also included as controls when performing the assay on a test sample (i.e., a sample in which the identity of the allele(s) at the PS is unknown). The specificity of an assay may also be confirmed by comparing the assay result for a test sample with the result obtained for the same sample using a different type of assay, such as by determining the sequence of an amplified target region believed to contain the PS of interest and comparing the determined sequence to context sequences accepted in the art, such as the context sequences provided herein.

The length of the context sequence necessary to establish that the correct genomic position is being assayed will vary based on the uniqueness of the sequence in the target region (for example, there may be one or more highly homologous sequences located in other genomic regions). The skilled artisan can readily determine an appropriate length for a context sequence for any PS using known techniques such as BLASTing the context sequence against publicly available sequence databases. For amplified target regions, which provide a first level of specificity, examining the context sequence of about 30 to 60 bases on each side of the PS in known samples is typically sufficient to ensure that the assay design is specific for the PS of interest. Occasionally, a validated assay may fail to provide an unambiguous result for a test sample. This is usually the result of the sample having DNA of insufficient purity or quantity, and an unambiguous result is usually obtained by repurifying or reisolating the DNA sample or by assaying the sample using a different type of assay.

For detecting PS characterized by an insertion/deletion variations, a number of assay techniques can be employed. Insertion/deletion variants can be detected by Sanger sequencing methods which employ di-deoxynucleosidetriphosphates. In some embodiments, commercially available software packages such as Mutation Surveyor® software available from SoftGenetics LLC, State College, Pa., USA that can detect homozygous and heterozygous insertion/deletion variants. In addition, the fragment analysis method disclosed in Hjelm et al. in The Journal of Molecular Diagnostics 12(5), pp 607-610 (2010) can be used to characterize insertion and deletion variants.

Programs such as Variant Caller with Multinomial Probabilistic Mode as disclosed in Scientific Reports, 3, 2161 (2013) and at http://emu.src.riken.jpNCMM/ can be used to detect insertion/deletion variants with high accuracy. Another method for the detection of insertion/deletion variants is disclosed in Z. Yhang et al., Nucleic Acids Research 43(9) 349 (2015), which relies on amplicon labeling and automated capillary electrophoresis.

Further, in performing any of the methods described herein that require determining the presence or absence of the Genetic Markers, such determination may be made by consulting a data repository that contains sufficient information on the patient's genetic composition to determine whether the patient has the marker. Preferably, the data repository lists whether the Genetic Markers are present and absent in the individual. The data repository could include the individual's patient records, a medical data card, a file (e.g., a flat ASCII file) accessible by a computer or other electronic or non-electronic media on which appropriate information or genetic data can be stored. As used herein, a medical data card is a portable storage device such as a magnetic data card, a smart card, which has an on-board processing unit and which is sold by vendors such as Siemens of Munich Germany, or a flash-memory card. If the data repository is a file accessible by a computer; such files may be located on various media, including: a server, a client, a hard disk, a CD, a DVD, a personal digital assistant such as a smart phone, a tape, a zip disk, the computer's internal ROM (read-only-memory) or the internet or worldwide web. Other media for the storage of files accessible by a computer will be obvious to one skilled in the art.

The invention also contemplates that testing for the Genetic Markers may be determined by investigating whether the individual has an allele, e.g., a particular nucleotide sequence, at a different locus that is in high linkage disequilibrium (LD) with the better response allele for the rs12748961 SNP or one of the other Genetic Markers identified in Table 1 above. Two particular alleles at different loci on the same chromosome are said to be in LD if the presence of one of the alleles at one locus tends to predict the presence of the other allele at the other locus. Such variants, which are referred to herein as linked variants, or proxy variants, may be any type of variant (e.g., a SNP, insertion or deletion variant) that is in high LD with the better response allele of interest.

Linked variants are readily identified by determining the degree of linkage disequilibrium (LD) between the better response allele of the rs12748961 SNP, for example, and a candidate linked allele. The candidate linked variant may be an allele of a polymorphism that is currently known. Other candidate linked variants may be readily identified by the skilled artisan using any technique well-known in the art for discovering polymorphisms.

The degree of LD between a better response allele in one of the Genetic Markers, e.g., the rs12748961 SNP, and a candidate linked variant may be determined using any LD measurement known in the art. LD patterns in genomic regions are readily determined empirically in appropriately chosen samples using various techniques known in the art for determining whether any two alleles (e.g., between nucleotides at different PSs) are in linkage disequilibrium (see, e.g., GENETIC DATA ANALYSIS II, Weir, Sineuer Associates, Inc. Publishers, Sunderland, Mass. 1996). The skilled artisan may readily select which method of determining LD will be best suited for a particular population sample size and genomic region. One of the most frequently used measures of linkage disequilibrium is r², which is calculated using the formula described by Devlin et al. (Genomics, 29(2):311-22 (1995)). r² is the measure of how well an allele X at a first locus predicts the occurrence of an allele Y at a second locus on the same chromosome. The measure only reaches 1.0 when the prediction is perfect (e.g., X if and only if Y).

In one embodiment, the locus of the linked variant is in a genomic region of about 100 kilobases, more preferably about 10 kb that spans any of the PS of the rs12748961 SNP. Other linked variants are those in which the LD with the better response allele has a r² value, as measured in a suitable reference population, of at least 0.75, more preferably at least 0.80, even more preferably at least 0.85 or at least 0.90, yet more preferably at least 0.95, and most preferably 1.0. The reference population used for this r² measurement may be the general population, a population using the CRTH2 receptor antagonist, a population diagnosed with a particular condition for which the CRTH2 receptor antagonist shows efficacy or a population whose members are self-identified as belonging to the same ethnic group, such as Caucasian, African American, Hispanic, Latino, Native American and the like, or any combination of these categories. Preferably the reference population reflects the genetic diversity of the population of patients to be treated with a CRTH2 receptor antagonist.

Example 2 of U.S. Patent Application Publication No. US20180237856, incorporated herein by reference in its entirety, discloses additional genetic variants other than rs12748961 which are predictive of a beneficial response to a CRTH2 receptor antagonist/montelukast combination. The r2 reported in Table 3 below is the Pearson correlation coefficient squared, where the Pearson correlation coefficient is calculated from the genotype data in Example 2 of the publication. The second column indicates the Pearson correlation squared with respect to presence of the C allele of the rs12748961 SNP. r2 ranges from 0 to 1, with 1 representing two perfectly correlated variants and 0 representing two independent variants (based on the analysis dataset).

TABLE 3

| | r² with |
|---|---|
| PS | rs12748961 |
| rs12748961 | 1.00 |
| rs12118655 | 0.93 |
| rs6679073 | 0.52 |
| rs12564209 | 1.00 |
| rs3805 | 0.97 |
| rs71633561 | 1.00 |
| rs71970505 | 1.00 |
| rs12132270 | 0.89 |
| rs67625805 | 0.91 |
| rs3747972 | 0.93 |
| rs11557080 | 0.96 |
| rs71633563 | 0.96 |
| rs34848415 | 0.09 |
| rs1891091 | 0.89 |

In some embodiments, a physician determines whether a patient has a Genetic Marker described herein by ordering a diagnostic test, which is designed to determine whether the patient has at least one copy of the better response allele of one of the Genetic Markers in Table 1, e.g., the rs12748961 SNP. Preferably the test determines the identity of both alleles, i.e., the genotype, at this PS. In some embodiments, the testing laboratory will prepare a nucleic acid sample from a biological sample (such as a blood sample or buccal swab) obtained from the patient. In some embodiments, a blood sample from the patient is drawn by the physician or a member of the physician's staff, or by a technician at a diagnostic laboratory. In some embodiments, the patient is provided with a kit for taking a buccal swab from the inside of her cheek, which the patient then gives to the physician's staff member or sends directly to the diagnostic laboratory.

In some embodiments, the testing laboratory does not know the identity of the individual whose sample it is testing; i.e., the sample received by the laboratory is made anonymous in some manner before being sent to the laboratory. For example, the sample may be merely identified by a number or some other code (a “sample ID”) and the results of the diagnostic method can be reported to the party ordering the test using the sample ID.

In some embodiments, after the test results have been obtained, the testing laboratory generates a test report which indicates whether the better response allele is present or absent at the genotyped polymorphic site, and preferably indicates whether the patient is heterozygous or homozygous for the better response allele. In some embodiments, the test report is a written document prepared by the testing laboratory and sent to the patient or the patient's physician as a hard copy or via electronic mail. In other embodiments, the test report is generated by a computer program and displayed on a video monitor in the physician's office. The test report may also comprise an oral transmission of the test results directly to the patient or the patient's physician or an authorized employee in the physician's office. Similarly, the test report may comprise a record of the test results that the physician makes in the patient's file.

In one embodiment, if the patient tests positive for at least one copy of the better response allele, then the test report further indicates that the patient tested positive for a genetic marker associated with a likely response to treatment with a CRTH2 antagonist, while if the individual tests negative for the better response allele, then the test report further indicates that the patient tested negative for a genetic marker associated with a likely response to treatment with a CRTH2 antagonist.

Typically, the individual would be tested for the presence of a Genetic Marker prior to initiation of the CRTH2 receptor antagonist therapy, but it is envisioned that such testing could be performed at any time after the individual is administered the first dose of a CRTH2 receptor antagonist. For example, the treating physician may be concerned that the patient has not responded adequately and desires to test the individual to determine whether continued treatment with the CRTH2 receptor antagonist is warranted. In some embodiments, a physician may determine whether or not an individual should be tested for a Genetic Marker. For example, the physician may be considering whether to prescribe for the patient a pharmaceutical product that is indicated for patients who test positive for the Genetic Marker.

In deciding how to use the Genetic Marker test results in treating any individual patient, the physician may also take into account other relevant circumstances, such as the disease or condition to be treated, the age, weight, gender, genetic background and race of the patient, including inputting a combination of these factors and the Genetic Marker test results into a model that helps guide the physician in choosing a therapy and/or treatment regimen with that therapy.

Detecting the presence or absence of any of the Genetic Markers may be performed using a kit that has been specially designed for this purpose. In one embodiment, a kit of the invention comprises a set of oligonucleotides designed for identifying each of the alleles at the PS, e.g., in rs12748961.

In some embodiments, the oligonucleotides in the kit are either allele-specific probes or allele-specific primers. In other embodiments, the kit comprises primer-extension oligonucleotides. In still further embodiments, the set of oligonucleotides is a combination of allele-specific probes, allele-specific primers and primer-extension oligonucleotides. The kit may comprise oligonucleotides designed for detecting the presence of other genetic markers associated with response to CRTH2 receptor antagonist therapy.

Oligonucleotides in kits of the invention must be capable of specifically hybridizing to a target region of a polynucleotide. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. In some embodiments, the target region contains the PS of interest, while in other embodiments, the target region is located one to 10 nucleotides adjacent to the PS.

The composition and length of each oligonucleotide in the kit will depend on the nature of the genomic region containing the PS as well as the type of assay to be performed with the oligonucleotide and is readily determined by the skilled artisan.

For example, the polynucleotide to be used in the assay may constitute an amplification product, and thus the required specificity of the oligonucleotide is with respect to hybridization to the target region in the amplification product rather than in genomic or cDNA isolated from the individual. As another example, if the kit is designed to genotype two or more polymorphic sites simultaneously, the melting temperatures for the oligonucleotides for each PS in the kit will typically be within a narrow range, preferably less than about 5° C. and more preferably less than about 2° C.

In some embodiments, each oligonucleotide in the kit is a perfect complement of its target region. An oligonucleotide is said to be a “perfect” or “complete” complement of another nucleic acid molecule if every nucleotide of one of the molecules is complementary to the nucleotide at the corresponding position of the other molecule. While perfectly complementary oligonucleotides are preferred for detecting polymorphisms, departures from complete complementarity are contemplated where such departures do not prevent the molecule from specifically hybridizing to the target region as defined above. For example, an oligonucleotide primer may have a non-complementary fragment at its 5′ end, with the remainder of the primer being completely complementary to the target region. Alternatively, non-complementary nucleotides may be interspersed into the probe or primer as long as the resulting probe or primer is still capable of specifically hybridizing to the target region.

In some preferred embodiments, each oligonucleotide in the kit specifically hybridizes to its target region under stringent hybridization conditions. Stringent hybridization conditions are sequence-dependent and vary depending on the circumstances. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. As the target sequences are generally present in excess, at Tm, 50% of the probes are occupied at equilibrium.

Typically, stringent conditions include a salt concentration of at least about 0.01 to 1.0 M sodium ion concentration (or other salts) at pH 7.0 to 8.3 and the temperature is at least about 25° C. for short oligonucleotide probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations. Additional stringent conditions can be found in Molecular Cloning: A Laboratory Manual, Sambrook et al., Cold Spring Harbor Press, Cold Spring Harbor, N.Y. (1989), chapters 7, 9, and 11, and in NUCLEIC ACID HYBRIDIZATION, A PRACTICAL APPROACH, Haymes et al., IRL Press, Washington, D.C., 1985.

One non-limiting example of stringent hybridization conditions includes hybridization in 4X sodium chloride/sodium citrate (SSC), at about 65-70° C. (or alternatively hybridization in 4× SSC plus 50% formamide at about 42-50° C.) followed by one or more washes in 1× SSC, at about 65-70° C. A non-limiting example of highly stringent hybridization conditions includes hybridization in 1× SSC, at about 65-70° C. (or alternatively hybridization in 1× SSC plus 50% formamide at about 42-50° C.) followed by one or more washes in 0.3× SSC, at about 65-70° C. A non-limiting example of reduced stringency hybridization conditions includes hybridization in 4× SSC, at about 50-60° C. (or alternatively hybridization in 6× SSC plus 50% formamide at about 40-45° C.) followed by one or more washes in 2× SSC, at about 50-60° C. Stringency conditions with ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25mM EDTA, pH 7.4) can be substituted for SSC (1× SSC is 0.15M NaCl and 15mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tm is determined according to the following equations. For hybrids less than 18 base pairs in length, Tₘ (° C.)=2(# of A+T bases)+4(# of G+C bases). For hybrids between 18 and 49 base pairs in length, Tₘ (° C.)=81.5+16.6(log₁₀[Na+])+0.41(% G+C)−(600/N), where N is the number of bases in the hybrid, and [Na+] is the concentration of sodium ions in the hybridization buffer ([Na+] for 1× SSC=0.165 M).

The oligonucleotides in kits of the invention may be comprised of any phosphorylation state of ribonucleotides, deoxyribonucleotides, and acyclic nucleotide derivatives, and other functionally equivalent derivatives. Alternatively, the oligonucleotides may have a phosphate-free backbone, which may be comprised of linkages such as carboxymethyl, acetamidate, carbamate, polyamide (peptide nucleic acid (PNA)) and the like (Varma, in MOLECULAR BIOLOGY AND BIOTECHNOLOGY, A COMPREHENSIVE DESK REFERENCE, Meyers, ed., pp. 6 17-20, VCH Publishers, Inc., 1995). The oligonucleotides may be prepared by chemical synthesis using any suitable methodology known in the art, or may be derived from a biological sample, for example, by restriction digestion. The oligonucleotides may contain a detectable label, according to any technique known in the art, including use of radiolabels, fluorescent labels, enzymatic labels, proteins, haptens, antibodies, sequence tags and the like. The oligonucleotides in the kit may be manufactured and marketed as analyte specific reagents (ASRs) or may constitute components of an approved diagnostic device.

In some embodiments, the set of oligonucleotides in the kit have different labels to allow simultaneous determination of the identity of the alleles at two or more polymorphic sites. The oligonucleotides may also comprise an ordered array of oligonucleotides immobilized on a solid surface such as a microchip, silica beads (such as BeadArray technology from Illumina, San Diego, Calif.), or a glass slide (see, e.g., WO 98/20020 and WO 98/20019). Kits comprising such immobilized oligonucleotides may be designed to perform a variety of polymorphism detection assays, including but not limited to probe hybridization and polymerase extension assays.

Kits of the invention may also contain other reagents such as hybridization buffer (e.g., where the oligonucleotides are to be used as allele-specific probes) or dideoxynucleotide triphosphates (ddNTPs; e.g., where the alleles at the polymorphic sites are to be detected by primer extension). Kits designed for use in polymerase-mediated genotyping assays, may also contain a polymerase and a reaction buffer optimized for the polymerase-mediated assay to be performed.

Kits of the invention may also include reagents to detect when a specific hybridization has occurred or a specific polymerase-mediated extension has occurred. Such detection reagents may include biotin-or fluorescent-tagged oligonucleotides or ddNTPs and/or an enzyme-labeled antibody and one or more substrates that generate a detectable signal when acted on by the enzyme.

It will be understood by the skilled artisan that the set of oligonucleotides and reagents for performing the assay will be provided in separate receptacles placed in the kit container if appropriate to preserve biological or chemical activity and enable proper use in the assay.

In other embodiments, each of the oligonucleotides and all other reagents in the kit have been quality tested for optimal performance in an assay designed to determine the genotype for at least one or more of the PS in Table 1 above, e.g., for the rs12748961 SNP. In some embodiments, the kit includes an instruction manual that describes how to use the determined genotype to assign, to the tested nucleic acid sample, the presence or absence of a response marker.

In some preferred embodiments, the set of oligonucleotides in the kit are allele-specific oligonucleotides. As used herein, the term allele-specific oligonucleotide (ASO) means an oligonucleotide that is able, under sufficiently stringent conditions, to hybridize specifically to one allele of a PS, at a target region containing the PS while not hybridizing to the same region containing a different allele. As understood by the skilled artisan, allele-specificity will depend upon a variety of readily optimized stringency conditions, including salt and formamide concentrations, as well as temperatures for both the hybridization and washing steps.

Examples of hybridization and washing conditions typically used for ASO probes and primers are found in Kogan et al., “Genetic Prediction of Hemophilia A” in PCR PROTOCOLS, A GUIDE TO METHODS AND APPLICATIONS, Academic Press, 1990, and Ruaflo et al., Proc. Nati. Acad. Sci. USA 87:6296-300 (1990).

Typically, an ASO will be perfectly complementary to one allele while containing a single mismatch for the other allele. In ASO probes, the single mismatch is preferably within a central position of the oligonucleotide probe as it aligns with the polymorphic site in the target region (e.g., approximately the 7th or 8th position in a 15mer, the 8th or 9th position in a 16mer, and the 10th or 11th position in a 20mer). The single mismatch in ASO primers is located at the 3′ terminal nucleotide, or preferably at the 3′ penultimate nucleotide. ASO probes and primers hybridizing to either the coding or noncoding strand are contemplated by the invention.

In some embodiments, the kit comprises a pair of allele-specific oligonucleotides for each PS to be assayed, with one member of the pair being specific for one allele (e.g., the better response allele) and the other member being specific for the other allele. In such embodiments, the oligonucleotides in the pair may have different lengths or have different detectable labels to allow the user of the kit to determine the genotype for the assayed PS.

In still other preferred embodiments, the oligonucleotides in the kit are primer-extension oligonucleotides. Termination mixes for polymerase-mediated extension from any of these oligonucleotides are chosen to terminate extension of the oligonucleotide at the PS of interest, or one base thereafter, depending on the alternative nucleotides present at the PS.

In another embodiment, the kit comprises a pair of allele specific oligonucleotide probes for genotyping at least one of the polymorphic sites in Table 1. In one embodiment, one ASO probe in the pair comprises a nucleotide sequence of at least 15 nucleotides that is identical to or perfectly complementary to the better response allele of the context sequence shown in Table 2 and the other ASO probe in the pair comprises a nucleotide sequence of at least 15 nucleotides that is identical to or perfectly complementary to the other allele of the context sequence shown in Table 2.

In another embodiment, the kit comprises a pair of allele specific oligonucleotide probes for genotyping the rs12748961 SNP. In one embodiment, one ASO probe in the pair comprises a nucleotide sequence of at least 15 nucleotides that is identical to or perfectly complementary to the better response allele of the rs12748961 SNP and the other ASO probe in the pair comprises a nucleotide sequence of at least 15 nucleotides that is identical to or perfectly complementary to the other allele of the the rs12748961 SNP.

In another embodiment, the kit comprises a pair of allele specific oligonucleotide probes for genotyping the rs12118655 SNP. In one embodiment, one ASO probe in the pair comprises a nucleotide sequence of at least 15 nucleotides that is identical to or perfectly complementary to the better response allele of the rs12118655 SNP and the other ASO probe in the pair comprises a nucleotide sequence of at least 15 nucleotides that is identical to or perfectly complementary to the other allele of the the rs12118655 SNP.

In another embodiment, the kit comprises a pair of allele specific oligonucleotide probes for genotyping the rs6679073 SNP. In one embodiment, one ASO probe in the pair comprises a nucleotide sequence of at least 15 nucleotides that is identical to or perfectly complementary to the better response allele of the rs6679073 SNP and the other ASO probe in the pair comprises a nucleotide sequence of at least 15 nucleotides that is identical to or perfectly complementary to the other allele of the the rs6679073 SNP.

In another embodiment, the kit comprises a pair of allele specific oligonucleotide probes for genotyping the rs12564209 SNP. In one embodiment, one ASO probe in the pair comprises a nucleotide sequence of at least 15 nucleotides that is identical to or perfectly complementary to the better response allele of the rs12564209 SNP and the other ASO probe in the pair comprises a nucleotide sequence of at least 15 nucleotides that is identical to or perfectly complementary to the other allele of the the rs12564209 SNP.

In another embodiment, the kit comprises a pair of allele specific oligonucleotide probes for genotyping the rs3805 SNP. In one embodiment, one ASO probe in the pair comprises a nucleotide sequence of at least 15 nucleotides that is identical to or perfectly complementary to the better response allele of the rs3805 SNP and the other ASO probe in the pair comprises a nucleotide sequence of at least 15 nucleotides that is identical to or perfectly complementary to the other allele of the the rs3805 SNP.

In another embodiment, the kit comprises a pair of allele specific oligonucleotide probes for genotyping the rs71633561 SNP. In one embodiment, one ASO probe in the pair comprises a nucleotide sequence of at least 15 nucleotides that is identical to or perfectly complementary to the better response allele of the rs71633561 SNP and the other ASO probe in the pair comprises a nucleotide sequence of at least 15 nucleotides that is identical to or perfectly complementary to the other allele of the the rs71633561 SNP.

In another embodiment, the kit comprises a pair of allele specific oligonucleotide probes for genotyping the rs71970505 SNP. In one embodiment, one ASO probe in the pair comprises a nucleotide sequence of at least 15 nucleotides that is identical to or perfectly complementary to the better response allele of the rs71970505 SNP and the other ASO probe in the pair comprises a nucleotide sequence of at least 15 nucleotides that is identical to or perfectly complementary to the other allele of the the rs71970505 SNP.

In another embodiment, the kit comprises a pair of allele specific oligonucleotide probes for genotyping the rs12132270 SNP. In one embodiment, one ASO probe in the pair comprises a nucleotide sequence of at least 15 nucleotides that is identical to or perfectly complementary to the better response allele of the rs12132270SNP and the other ASO probe in the pair comprises a nucleotide sequence of at least 15 nucleotides that is identical to or perfectly complementary to the other allele of the the rs12132270 SNP.

In another embodiment, the kit comprises a pair of allele specific oligonucleotide probes for genotyping the rs67625805 SNP. In one embodiment, one ASO probe in the pair comprises a nucleotide sequence of at least 15 nucleotides that is identical to or perfectly complementary to the better response allele of the rs67625805 SNP and the other ASO probe in the pair comprises a nucleotide sequence of at least 15 nucleotides that is identical to or perfectly complementary to the other allele of the the rs67625805 SNP.

In another embodiment, the kit comprises a pair of allele specific oligonucleotide probes for genotyping the rs3747972 SNP. In one embodiment, one ASO probe in the pair comprises a nucleotide sequence of at least 15 nucleotides that is identical to or perfectly complementary to the better response allele of the rs3747972 SNP and the other ASO probe in the pair comprises a nucleotide sequence of at least 15 nucleotides that is identical to or perfectly complementary to the other allele of the the rs3747972 SNP.

In another embodiment, the kit comprises a pair of allele specific oligonucleotide probes for genotyping the rs11557080 SNP. In one embodiment, one ASO probe in the pair comprises a nucleotide sequence of at least 15 nucleotides that is identical to or perfectly complementary to the better response allele of the rs11557080 SNP and the other ASO probe in the pair comprises a nucleotide sequence of at least 15 nucleotides that is identical to or perfectly complementary to the other allele of the the rs11557080 SNP.

In another embodiment, the kit comprises a pair of allele specific oligonucleotide probes for genotyping the rs71633563 SNP. In one embodiment, one ASO probe in the pair comprises a nucleotide sequence of at least 15 nucleotides that is identical to or perfectly complementary to the better response allele of the rs71633563 SNP and the other ASO probe in the pair comprises a nucleotide sequence of at least 15 nucleotides that is identical to or perfectly complementary to the other allele of the the rs71633563 SNP.

In another embodiment, the kit comprises a pair of allele specific oligonucleotide probes for genotyping the rs34848415 SNP. In one embodiment, one ASO probe in the pair comprises a nucleotide sequence of at least 15 nucleotides that is identical to or perfectly complementary to the better response allele of the rs34848415 SNP and the other ASO probe in the pair comprises a nucleotide sequence of at least 15 nucleotides that is identical to or perfectly complementary to the other allele of the the rs34848415 SNP.

In another embodiment, the kit comprises a pair of allele specific oligonucleotide probes for genotyping the rs1891091 SNP. In one embodiment, one ASO probe in the pair comprises a nucleotide sequence of at least 15 nucleotides that is identical to or perfectly complementary to the better response allele of the rs1891091 SNP and the other ASO probe in the pair comprises a nucleotide sequence of at least 15 nucleotides that is identical to or perfectly complementary to the other allele of the the rs1891091 SNP.

C. Pharmaceutical Compositions, Drug Products and Treatment Regimens

An individual to be tested in, or treated by, any of the methods and products described herein is a human subject in need of treatment with a CRTH2 receptor antagonist. In some embodiments, the individual has been diagnosed with, or exhibits a symptom of, a disease susceptible to treatment with a CRTH2 receptor antagonist. In other embodiments, the CRTH2 receptor antagonist drug to be used has been approved for use in treating an indication with which the individual has been diagnosed.

In some embodiments, the CRTH2 receptor antagonist used in the pharmaceutical compositions, drug products, kits methods, and uses of the present invention may be any known CRTH2 receptor antagonist.

In one embodiment, the CRTH2 receptor antagonist is the compound of the formula I as disclosed in U.S. Pat. No. 8,394,819, the disclosure of which is hereby incorporated by reference as if fully set forth herein. This patent discloses a compound of formula I

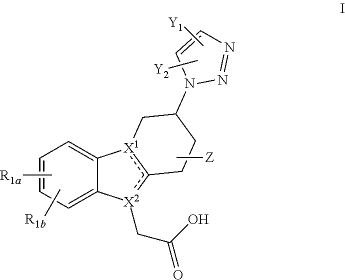

or a pharmaceutically acceptable salt thereof, wherein:

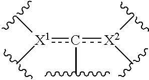

represents either

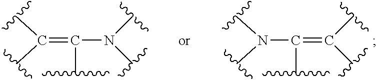 [SMILES: *C(*)=C(*)N(*)* ; *C(*)=C(*)N(*)*]

Y₁ is selected from optionally substituted aryl and —C(R₂)(R₃)(R₄);
Y₂ is selected from H and C₁₆alkyl;
Z is selected from H and C₁₆alkyl;
R₁ₐ and R_{1b} are independently selected from H, halogen, —OC₁₋₆alkyl, —O-haloC₁₋₆alkyl, C₁₋₆alkyl, haloC₁₋₆alkyl, optionally substituted aryl and (C₁₃alkylene)-optionally subsituted aryl;
R₂ is selected from H; —C₁₋₆alkyl optionally substituted with halogen, —OH or
—NHSO₂CH₃; —OH; OC₁₋₆alkyl; —S(O)ₙC₁₋₆alkyl; —CN; optionally substituted aryl; optionally substituted —O-aryl and optionally substituted heteroaryl, wherein n is 0, 1 or 2;
R₃ is selected from H, —C₁₋₆alkyl, C₁₋₆haloalkyl, optionally substituted aryl and optionally substituted heteroaryl; and
R₄ is selected from H, —C₁₋₆alkyl, C₁₋₆haloalkyl, optionally substituted aryl and optionally substituted heteroaryl; or
R₃, R₄ and the carbon atom to which they are attached together form —C₃₋₆cycloalkyl, fluorenyl or C₃₆heterocyclyl having a ring heteroatom selected from —N(R^{a})—, —O— and —S—; or
R₃, R₄ together represent C₁₋₆alkylidene;
R^{a} is H, C₁₋₆alkyl or —C(O)C₁₋₆alkyl; and
the optional substituent for aryl and heteroaryl is 1 to 4 groups independently selected from halogen, —C₁₋₃alkoxy, —C₁₋₃haloalkyl, hydroxy-C₁₋₃alkyl, —S(O)ₙ—C₁₋₃alkyl, amino, and mono- and di-(C₁₋₃alkyl)amino.

In specific embodiments, the CRTH2 receptor antagonist is {(7R)-4-fluoro-7-[5-(4-fluorobenzyl)-1H-[1,2,3]triazol-1-yl]-6,7,8,9-tetrahydropyrido[1,2-a]indol-10-yl}-acetic acid or a pharmaceutically acceptable salt thereof.

In other embodiments, the CRTH2 receptor antagonist is an antagonist disclosed in U.S. Pat. No. 8,592,383, the disclosure of which is hereby incorporated by reference as if fully set forth herein. In specific embodiments, the CRTH2 receptor antagonist is selected from:

4-{cyclopropyl[cis, cis-4-{[4-(trifluoromethoxy)phenyl]carbonyl}-2,3,3a,4,9,9a-hexahydro-1H-cyclopenta[b]quinolin-9-yl]amino}-4-oxobutanoic acid,

4-[cyclopropyl[cis,cis-7-fluoro-2,3,3a,4,9,9a-hexahydro-4-[4-(trifluoromethoxy)benzoyl]-1H-cyclopenta[b]quinolin-9-yl]amino]-4-oxobutanoic acid,

4-(cyclopropyl((3aS,9R,9aR)-7-fluoro-4-(4-(trifluoromethoxy)benzoyl)-2,3,3a,4,9,9a-hexahydro-1H-cyclopenta[b]quinolin-9-yl)amino)-4-oxobutanoic acid,

4-[cyclopropyl[cis,cis-1,2,2a,3,8,8a-hexahydro-3-[4-(trifluoromethoxy)benzoyl]cyclobuta[b]quinolin-8-yl]amino]-4-oxobutanoic acid,

(R)-1-((cis,cis-3-(benzyloxycarbonyl)-5,6-difluoro-1,2,2a,3,8,8a-hexahydrocyclobuta[b]quinolin-8-yl)(cyclopropyl)carbamoyl)azetidine-2-carboxylic acid, or a pharmaceutically acceptable salt thereof.

In another embodiment, the compound is 2-(2-methyl-1-(4-(methylsulfonyl)-2-(trifluoromethyl)benzyl)-1H-pyrrolo[2,3-b]pyridin-3-yl)acetic acid (fevipiprant), or a pharmaceutically acceptable salt thereof as disclosed in U.S. Pat. No. 7,666,878.

In another embodiment, the compound is 3-((3R)-3-{[(4-fluorophenyl)sulfonyl]amino}-1,2,3,4-tetrahydro-9H-carbazol-9-yl)propanoic acid (ramatroban) or a pharmaceutically acceptable salt thereof

In some embodiments, the CRTH2 receptor antagonist is administered in combination with a leukotriene receptor antagonist, such as montelukast, zafilukast, or pranlukast. In specific embodiments, the leukotriene receptor antagonist is montelukast. The CRTH2 receptor antagonist and leukotriene receptor antagonist can be administered in the same or separate dosage forms.

In specific embodiments of the combination therapy, the CRTH2 antagonist is {(7R)-4-fluoro-7-[5-(4-fluorobenzyl)-1H-[1,2,3]triazol-1-yl]-6,7,8,9-tetrahydropyrido[1,2-a]indol-10-yl}-acetic acid or a pharmaceutically acceptable salt thereof, and the leukotriene receptor antagonist is montelukast. In other specific embodiments, the CRTH2 receptor antagonist is fevipiprant or a pharmaceutically acceptable salt thereof and the leukotriene receptor antagonist is montelukast.

Disorders that may be treated with the pharmaceutical compositions, drug products, kits, methods, and uses of the present invention in accordance with the present invention are generally those that are susceptible to treatment with CRTH2 receptor antagonist therapy, i.e., the CRTH2 receptor antagonist achieves a clinically measurable beneficial result in a group of patients with the disease. Exemplary diseases and conditions susceptible to treatment with a CRTH2 receptor antagonist include but are not limited to diseases include asthma, congestion, allergic rhinitis, atopic dermatitis, chronic obstructive pulmonary disease (COPD), dermatitis, inflammatory bowel disease, rheumatoid arthritis, allergic nephritis, conjunctivitis, bronchial asthma, food allergy, systemic mast cell disorder, anaphylactic shock, urticaria, eczema, itching, inflammation, ischemia-reperfusion injury, cerebrovascular disorders, pleuritis, ulcerative colitis, eosinophil-related diseases, such as eosinophilic granulomatosis with polyangiitis (EGPA) and sinusitis, and basophil-related diseases, such as basophilic leukemia and basophilic leukocytosis, in humans and other mammals. Examples of cerebrovascular disorders include stroke.

In certain embodiments, the present invention provides a pharmaceutical composition, drug product, kit, method, or use for treating asthma, congestion, allergic rhinitis or COPD which include instructions for administering a therapeutically effective dose of CRTH2 receptor antagonist to a patient in need of such treatment. In a specific embodiment, the disease or condition being treated is asthma. In another embodiment, the disease or condition being treated is COPD.

In addition, the CRTH2 receptor antagonists can inhibit prostanoid-induced smooth muscle contraction by antagonizing contractile prostanoids or mimicking relaxing prostanoids and hence may be used in the inventive methods of treatment for dysmenorrhea, premature labor and eosinophil-related disorders.

In preferred embodiments, the methods of the present invention are used in conjunction with any CRTH2 receptor antagonist monotherapy or combination therapy treatment regimen comprising a CRTH2 receptor antagonist and a leukotriene receptor antagonist, e.g., montelukast, for treating asthma, including allergic asthma.

The doses and dosage regimen of the other agents used in the combination therapies of the present invention for the treatment of disorders susceptible to treatment by a CRTH2 antagonist can be determined by the attending clinician, taking into consideration the approved doses and dosage regimen in the package insert; and the age, sex and general health of the patient. Agents administered in combination therapy can be administered simultaneously (i.e., in the same composition or in separate compositions one right after the other) or sequentially. This is particularly useful when the components of the combination are given on different dosing schedules, e.g., one component is administered once daily and another every six hours, or when the preferred pharmaceutical compositions are different, e.g., one is a tablet and one is a capsule. A kit comprising the separate dosage forms is therefore advantageous.

When administering a combination therapy that is selected to treat a patient based on the presence or absence of the blood biomarker and Genetic Marker in the patient, the therapeutic agents in the combination, or a pharmaceutical composition or compositions comprising the therapeutic agents, may be administered in any order such as, for example, sequentially, concurrently, together, simultaneously and the like. The amounts of the various therapeutic agents in such combination therapy may be different amounts (different dosage amounts) or same amounts (same dosage amounts). In some embodiments, the agents in the combination are administered in doses commonly employed when such agents are used as monotherapy for treating the patient's disease or condition, while in other embodiments, the agents are administered in doses lower than the doses commonly employed when such agents are used as monotherapy for treating the disease or condition.

In some embodiments, the therapeutic agents used in combination therapy are present in the same pharmaceutical composition, which may be suitable for oral administration, intravenous administration, subcutaneous administration or parenteral administration.

In a specific embodiment, the therapeutic agents used in combination therapy are present in the same pharmaceutical composition, which is a tablet suitable for oral administration.

The inventors herein also contemplate that the blood biomarker and Genetic Markers described herein could be used to seek regulatory approval to market a new CRTH2 receptor antagonist drug product for an indication that includes a disease component, a blood biomarker component, and a Genetic Marker component. The disease component is a disease susceptible to treatment with the CRTH2 receptor antagonist, the blood biomarker is a patient whose blood eosinophil count is at least 250 eosinophils/microliter; and the genetic marker component is a patient who tests positive for at least one copy of one of the better response alleles as set forth in Table 1 (e.g., for at least one of one copy of the C allele of the rs12748961 SNP). Similarly, the inventors herein contemplate that the combination of the blood biomarker and Genetic Marker is useful for seeking approval of such indications for currently approved CRTH2 receptor antagonist drugs that physicians are reluctant to prescribe for certain diseases based on the marginal benefit/risk ratio of the drug for such diseases in the general population.

Seeking approval for a indication typically involves measuring the incidence of a desired response to a drug in separate groups of patients treated with the drug. Each individual within one of the groups has disease, blood biomarker and genetic profiles that place the individual within the proposed indication. The individuals in the other group may be randomly selected without regard to whether they have the blood biomarker or genetic marker component of the proposed indication. The drug product for which approval is sought could be administered to the groups in a prospective trial. Alternatively, a retrospective biomarker and pharmacogenetic analysis of patients previously treated with the drug could be performed.

The drug product for which an indication is being sought could be evaluated with other therapeutically active agents, for example another drug with efficacy for treating the disease or condition in the proposed indication or an agent that is intended to reduce the incidence of an adverse effect caused by the drug. In some embodiments, the indication for which regulatory approval is sought may include other markers (other biomarkers or genetic markers) or predictors of response to the drug.

The study could be designed in consultation with representatives of the regulatory agency or government entity from whom approval is required before marketing the drug product in a particular country. Preferably, the regulatory agency is authorized by the government of a major industrialized country, such as Australia, Canada, China, a member of the European Union, Japan, South Korea, Taiwan and the like. Most preferably the regulatory agency is authorized by the government of the United States and the type of application for approval that is filed will depend on the legal requirements set forth in the last enacted version of the Food, Drug and Cosmetic Act that are applicable for the drug product and may also include other considerations such as the cost of making the regulatory filing and the marketing strategy for the drug product. For example, if the pharmaceutical formulation in the drug product has previously been approved for the disease component of the proposed biomarker and pharmacogenetic indication, then the application might be a paper NDA, a supplemental NDA or an abbreviated NDA, but the application might need to be a full NDA if the pharmaceutical formulation has never been approved before; with these terms having the meanings applied to them by those skilled in the pharmaceutical arts or as defined in the Drug Price Competition and Patent Term Restoration Act of 1984.

One desired outcome of a clinical trial using the combination of the blood biomarker and Genetic Markers described herein is approval to market a drug product which comprises (1) a CRTH2 receptor antagonist pharmaceutical composition and (2) prescribing information which includes a biomarker and pharmacogenetic indication for which the pharmaceutical composition is recommended. Prescribing information is typically found in the product insert, also frequently referred to as the package insert or label, for the drug.

As discussed above, the proposed indication has three components: a disease component, a blood biomarker component, and the Genetic Marker component. Thus, the prescribing information would describe a defined group of patients for which the drug has demonstrated efficacy for one or more diseases, symptoms or medical conditions. In some embodiments, the prescribing information will discuss how to identify individuals who are in the defined group. For example, in some embodiments, the prescribing information states that the drug is indicated for individuals who have a blood eosinophil count of at least 250 cells/microliter, who test positive for the better response allele of a Genetic Marker described herein. Alternately, the prescribing information may state that the drug is contraindicated for individuals who have a blood eosinophil count of less than 250 cells/microliter or who test negative for a better response allele of a Genetic Marker described herein. In some preferred embodiments, the prescribing information includes the name of at least one approved diagnostic test to be used for detecting the presence or absence of the required Genetic Marker component of the indication. The proposed indication in a drug product of the invention may include r a requirement to use the drug in combination with one or more other therapeutically active agents (e.g., montelukast).

The prescribing information may include information on recommended dosages and treatment regimens.

In preferred drug compositions used in the invention, the pharmaceutical composition comprises a CRTH2 receptor antagonist selected from {(7R)-4-fluoro-7-[5-(4-fluorobenzyl)-1H-[1,2,3]triazol-1-yl]-6,7,8,9-tetrahydropyrido[1,2-a]indol-10-yl}-acetic acid and fevipiprant.

More preferably, the pharmaceutical composition comprises a CRTH2 receptor antagonist which is {(7R)-4-fluoro-7-[5-(4-fluorobenzyl)-1H-[1,2,3]triazol-1-yl]-6,7,8,9-tetrahydropyrido[1,2-a]indol-10-yl}-acetic acid and a leukotriene receptor antagonist such as montelukast. A preferred indication for the drug products of the invention comprises the use of the pharmaceutical composition for the treatment of patients suffering from asthma, who have a blood eosinophil count of at least 250 cells/microliter, and at least one copy of one of the better response alleles for the Genetic Markers set forth in Table 1. In preferred embodiments, the patients test positive for at least one copy of the C allele for the rs12748961 SNP. In other preferred embodiments, the patients have a blood eosinophil count of at least 300 cells/microliter and also test positive for at least one copy of the C allele for the rs12748961 SNP. In some embodiments, the prescribing information states that the CRTH2 receptor antagonist pharmaceutical composition is indicated in combination with a leukotriene receptor antagonist (e.g., montelukast) for treating patients suffering from asthma.

Any or all analytical and mathematical operations involved in performing the methods and uses described herein or in using the kits, composition and drug products described herein may be implemented by a computer. For example, the computer may execute a computer program that assigns the blood eosinophil count and the presence or absence of the better response allele of the Genetic Marker to an individual based on data inputted by an employee of a testing laboratory or by the treating physician. In addition, the same computer or a different computer may output the predicted response to CRTH2 receptor antagonist therapy based on that blood eosinophil count and Genetic Marker assignment. Data relating to the blood eosinophil count and the presence or absence of the better response allele of a Genetic Marker in an individual may be stored as part of a relational database (e.g., an instance of an Oracle database or a set of ASCII flat files) containing other clinical and/or genetic data for the individual. These data may be stored on the computer's hard drive or may, for example, be stored on a CD ROM or on one or more other storage devices accessible by the computer. For example, the data may be stored on one or more databases in communication with the computer via a network.

### EXAMPLES

The following example is provided to more clearly describe the present invention and should not be construed to limit the scope of the invention.

### Example 1

### Clinical Study to Identify Patient Subgroup with Improved Lung Function with Treatment with a CRTH2 Receptor Antagonist in Subjects with Persistent Asthma that is Uncontrolled While Receiving Montelukast

The inventors conducted a world-wide, randomized, placebo-controlled, double-blind, parallel group study of the CRTH2 receptor antagonist, {(7R)-4-fluoro-7-[5-(4-fluorobenzyl)-1H-[1,2,3]triazol-1-yl]-6,7,8,9-tetrahydropyrido[1,2-a]indol-10-yl}-acetic acid (hereinafter “Compound A”) in participants with persistent asthma who remained uncontrolled while being maintained on montelukast (ML). At Prescreening Visit 1, participants were tested for the presence of the rs 12748961 SNP to determine eligibility. The primary efficacy analysis compared the effect of Compound A to placebo, when each was added to montelukast, on the average change from baseline in FEV1 using a constrained longitudinal data analysis model.

### Summary of the Clinical Study Design

Eligible participants were male and female participants between the ages of 18 and 65 with persistent asthma for at least 1 year and a documented SNP+test result. Participants also must have had acceptable lung function at Visit 2 and Visit 4 and evidence of reversibility of airway obstruction. Approximately 139 eligible participants whose asthma was uncontrolled during a run-in period of approximately 2 to 4 weeks entered a 6-week double-blind treatment period and were randomized in a 1:1 ratio to receive either Compound A 150 mg and montelukast 10 mg or Compound A 150 mg matching-image placebo and montelukast 10 mg. Randomization was stratified according to the number of C alleles (having either 1 or 2 copies of the allele) at the rs12748961 SNP. The total study duration for each participant was up to 17 weeks.

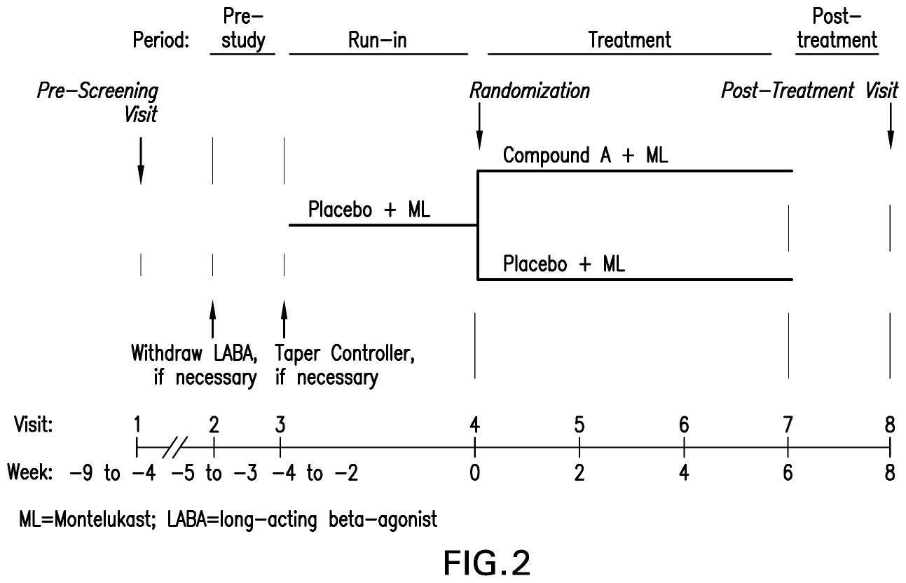

FIG. 2 is a graphical depiction of the study design. Briefly, salient activities occurring during the study are described below. During a pre-screening visit, a blood sample is collected to assess for the presence of the C-allele of rs12748961 SNP. At the start of the pre-study period, eligible subjects receiving Long-Acting Beta-Agonist (LABA) therapy were asked to discontinue the LABA. During run-in period (approximately 2 to 4 weeks), subjects receive single-blind Compound A 150 mg matching-image placebo and open-label montelukast 10 mg. Subjects taking asthma controller therapies prior to Visit 2 were asked to discontinue or taper off these pre-study medications before randomization while receiving open-label montelukast 10 mg. During the treatment period (approximately 6 weeks): Subjects received double-blind treatment of Compound A 150 mg plus montelukast 10 mg; or Compound A 150 mg matching-image placebo plus montelukast 10 mg.

### Results

When added to montelukast, treatment with Compound A was superior to placebo in increasing lung function in the full study population, as demonstrated by an increase in the average change from baseline of FEV₁ (L) at the end of Week 4 and Week 6 (0.107 L [0.015, 0.199]) (p=0.023). More detailed results for the full study population are shown in the Table 4 below.

TABLE 4

| | | FEV₁ (L) | | | | Average Change from Baseline in FEV₁ (L) | | | | |
|---|---|---|---|---|---|---|---|---|---|---|
| | | | | Average at Week | | at Week 4 and Week 6 | | | | |
| | | Baseline | | 4 and Week 6 | | | | | LS | |
| Treatment | N‡ | Mean | SD | Mean | SD | Median | Mean | SD | Mean | 95% CI |
| Cmpd A | 70 | 2.264 | 0.566 | 2.426 | 0.657 | 0.091 | 0.156 | 0.286 | 0.152 | (0.088, 0.217) |
| 150 mg + ML | | | | | | | | | | |
| Placebo + | 69 | 2.234 | 0.612 | 2.303 | 0.723 | 0.056 | 0.043 | 0.261 | 0.046 | (−0.020, 0.111) |
| ML | | | | | | | | | | |

| Estimated Differences with placebo + ML | Difference in LS Means§ | 95% CI | p-Value |
|---|---|---|---|
| Cmpd A 150 mg + ML vs. placebo + | 0.107 | (0.015, 0.199) | 0.023 |
| ML at Week 4 and Week 6 | | | |

†Constrained longitudinal data analysis model with terms for treatment, time, and the interaction of time by treatment, number of C alleles at the rs'961 SNP (1 copy/2 copies), and prior inhaled corticosteroids use (Yes/No).
‡Number of subjects included in the cLDA model.
§Positive differences are in favor of the Cmpd A 150 mg + ML treatment group in the comparison.
ML = montelukast;
Cmpd A = Compound A
SD = Standard Deviation;
CI = Confidence Interval.
Pooled SD = 0.271

The clinical study was intended to show efficacy of Compound A, when added to montelukast, for treatment of asthma in a population that was selected for entry into the study based on the presence of the C allele at the rs12748961 SNP. Based on this genetic marker, it was anticipated that this study would demonstrate the utility of a highly specific predictor of a clinically meaningful response to treatment with Compound A for asthma.

In order to identify patient groups susceptible to treatment response, the inventors conducted an analysis on pre-specified patient subgroups in the study based on (a) demographic variables (including age, gender, race, weight, height, and body mass index) and (b) baseline characteristics (including duration of asthma, age diagnosed of asthma, asthma controller pre- trial, ICS use, blood eosinophil count, baseline ACQ-6, and smoking history), (c) prior and secondary diagnoses, (d) prior and concomitant therapies, and (e) the number of C alleles of the rs12748961 SNP on asthmatic study subjects who had undergone a clinical trial with a CRTH2 receptor antagonist and the leukotriene antagonist montelukast. The analysis sought to assess whether a mean treatment difference varies across patient subgroups defined by several pre-specified characteristics and who also tested positive for at least one copy of the C allele of the rs12748961 SNP.

Upon examination of pre-specified patient subgroups using the criteria described in (a)-(e) for treatment differences, a much greater treatment difference of 0.193 L (193 mL) for FEV₁ was observed in a key pre-specified subgroup of participants with elevated peripheral blood eosinophil count at baseline (defined as at least 300 eosinophils per μL (i.e., ≥0.30×10⁹/L). More detailed results for this subgroup are shown in the Table 5 below.

TABLE 5

| | | | | Average Change | | | |
|---|---|---|---|---|---|---|---|
| | | Baseline FEV₁ | | From Baseline | | Estimated Differences | |
| | | (L) | | FEV₁ (L) | | With Placebo + ML | |
| Treatment | N | Mean | SD | Mean | SD | LS Mean | 95% CI |
| >0.30 × 10⁹/L* | | | | | | | |
| Cmpd A 150 mg + ML | 22 | 2.147 | 0.502 | 0.139 | 0.179 | 0.193 | (0.084, 0.301) |
| Placebo + ML | 19 | 2.302 | 0.711 | −0.059 | 0.151 | | |
| <0.30 × 10⁹/L* | | | | | | | |
| Cmpd A 150 mg + ML | 45 | 2.334 | 0.598 | 0.166 | 0.330 | 0.063 | (−0.061, 0.188) |
| Placebo + ML | 46 | 2.207 | 0.590 | 0.086 | 0.288 | | |

ML = montelukast;
Cmpd A = Compound A
SD = Standard Deviation;
Baseline was defined at Week 0. If Week 0 measurement was not available, the last non-missing value before treatment was used as Baseline.
N = The number of subjects in each treatment group that has non-missing change from baseline value at particular weeks.
*Refers to the baseline eosinophil count

This subgroup constituted less than one-third of the population in this study. In the remaining approximately 70% of the study population, the FEV₁ effect size showed a difference of 0.063L (63 mL). This pre-specified subgroup analysis indicates that robust efficacy of the CRTH2 antagonist, Compound A, when added to montelukast, can be shown in a subpopulation of asthmatics selected based on the presence of the rs12748961 SNP genetic marker and an elevated eosinophil count at baseline.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

1. A method of treating a patient with a disorder susceptible to treatment with a CRTH2 receptor antagonist comprising:
administering a therapeutically effective amount of the CRTH2 receptor antagonist to the patient,
wherein said patient, prior to the administration of the CRTH2 receptor antagonist, has:
(a) a blood eosinophil count of at least 250 eosinophils/microliter; and
(b) tested positive for at least one copy of a better response allele selected from a Genetic Marker in the table below or a linked variant of one or more of the Genetic Markers:

| | | Better |
|---|---|---|
| Polymorphic | | Response |
| Site (PS) | Alleles | Allele |
| rs12748961 | T/C | C |
| rs12118655 | A/G | G |
| rs6679073 | C/A | A |
| rs12564209 | C/G | G |
| rs3805 | T/G/A | G |
| rs71633561 | G/C | C |
| rs71970505 | ATGCAGACTGT/— | — |
| rs12132270 | C/T | T |
| rs67625805 | T/— | — |
| rs3747972 | A/G | A |
| rs11557080 | G/A | A |
| rs71633563 | C/T | T |
| rs34848415 | A/— | — |
| rs1891091 | A/G | A. |

2. The method of claim 1, wherein the CRTH2 receptor antagonist response marker is the rs12748961 SNP, and
said patient, prior to the administration of the CRTH2 receptor antagonist, has tested positive for at least one copy of the C allele of the rs12748961 SNP.

3. The method of claim 1, wherein a leukotriene receptor antagonist is administered to the patient in addition to the CRTH2 receptor antagonist.

4. A method of diagnosing in a patient who is susceptible to treatment with a CRTH2 receptor antagonist and treating asthma, said method comprising:
(a) obtaining a blood sample from the patient and determining whether the patient's blood eosinophil count is at least 250 eosinophils/microliter;
(b) obtaining a biological sample from the patient and detecting whether a better response allele of at least one of the Genetic Markers in the table below or a linked variant of one or more of the Genetic Markers is present in the biological sample;

| | | Better |
|---|---|---|
| Polymorphic | | Response |
| Site (PS) | Alleles | Allele |
| rs12748961 | T/C | C |
| rs12118655 | A/G | G |
| rs6679073 | C/A | A |
| rs12564209 | C/G | G |
| rs3805 | T/G/A | G |
| rs71633561 | G/C | C |
| rs71970505 | ATGCAGACTGT/— | — |
| rs12132270 | C/T | T |
| rs67625805 | T/— | — |
| rs3747972 | A/G | A |
| rs11557080 | G/A | A |
| rs71633563 | C/T | T |
| rs34848415 | A/— | — |
| rs1891091 | A/G | A. |

(c) diagnosing the patient as susceptible to treatment with a CRTH2 receptor antagonist when the blood eosinophil count is determined to be at least 250 eosinophils/microliter and the presence of the better response allele in the biological sample is detected; and
(d) administering a therapeutically effective amount of a CRTH2 receptor antagonist to the diagnosed patient.

5. The method of claim 4, wherein
in step (b), the C allele of the CRTH2 receptor antagonist response marker is rs12748961 SNP is detected, and
in step (d), the patient is diagnosed as susceptible to treatment with a CRTH2 receptor antagonist when the blood eosinophil count is determined to be at least 250 eosinophils/microliter and the C allele of the rs12748961 SNP is detected.

6. The method of claim 4 or 5, wherein a leukotriene receptor antagonist is administered to the patient in addition to the CRTH2 receptor antagonist.

7. A drug product which comprises a pharmaceutical composition and prescribing information,
wherein the pharmaceutical composition comprises a CRTH2 receptor antagonist and the prescribing information comprises a blood eosinophil count and a pharmacogenetic indication,
wherein the prescribing information comprises the treatment of a disease susceptible to treatment with the CRTH2 receptor antagonist in patients who have a blood eosinophil count of at least 250 eosinophils/microliter and who also have pharmacogenetic indication, wherein said patients also test positive for at least one copy of a better response allele selected from a Genetic Marker in the table below or a linked variant of one or more of the Genetic Markers:

| | | Better |
|---|---|---|
| Polymorphic | | Response |
| Site (PS) | Alleles | Allele |
| rs12748961 | T/C | C |
| rs12118655 | A/G | G |
| rs6679073 | C/A | A |
| rs12564209 | C/G | G |
| rs3805 | T/G/A | G |
| rs71633561 | G/C | C |
| rs71970505 | ATGCAGACTGT/— | — |
| rs12132270 | C/T | T |
| rs67625805 | T/— | — |
| rs3747972 | A/G | A |
| rs11557080 | G/A | A |
| rs71633563 | C/T | T |
| rs34848415 | A/— | — |
| rs1891091 | A/G | A. |

8. The drug product of claim 7, wherein the prescribing information comprises the treatment of the disease susceptible to treatment with the CRTH2 receptor antagonist in patients who have a blood eosinophil count of at least 250 eosinophils/microliter and who also test positive for at least one copy of the C allele of the rs12748961 SNP.

9. The drug product of claim 7, wherein the pharmaceutical composition further comprises a leukotriene receptor antagonist.

10. The method or drug product of claim 1, wherein the disorder susceptible to treatment with the CRTH2 receptor antagonist is asthma.

11. The method or drug product of claim 1, wherein the CRTH2 receptor antagonist is {(7R)-4-fluoro-7-[5-(4-fluorobenzyl)-1H-[1,2,3]triazol-1-yl]-6,7,8,9-tetrahydropyrido[1,2-a]indol-10-yl}-acetic acid or 2-(2-methyl-1-(4-(methylsulfonyl)-2-(trifluoromethyl)benzyl)-1H-pyrrolo[2,3-b]pyridin-3-yl)acetic acid (fevipiprant), or a pharmaceutically acceptable salt thereof.

12. The method or drug product of claim 11, wherein the CRTH2 receptor antagonist is {(7R)-4-fluoro-7-[5-(4-fluorobenzyl)-1H-[1,2,3]triazol-1-yl]-6,7,8,9-tetrahydropyrido[1,2-a]indol-10-yl}-acetic acid or a pharmaceutically acceptable salt thereof.

13. The method of any one of claim 3, wherein the leukotriene receptor antagonist is montelukast.

14. The method of claim 1, wherein the blood eosinophil count is at least 300 eosinophils/microliter.

15. The method of claim 3, wherein the CRTH2 receptor antagonist is {(7R)-4-fluoro-7-[5-(4-fluorobenzyl)-1H-[1,2,3]triazol-1-yl]-6,7,8,9-tetrahydropyrido[1,2-a]indol-10-yl}-acetic acid or 2-(2-methyl-1-(4-(methylsulfonyl)-2-(trifluoromethyl)benzyl)-1H-pyrrolo[2,3-b]pyridin-3-yl)acetic acid (fevipiprant), or a pharmaceutically acceptable salt thereof.

16. The method of claim 15, wherein the CRTH2 receptor antagonist is {(7R)-4-fluoro-7-[5-(4-fluorobenzyl)-1H-[1,2,3]triazol-1-yl]-6,7, 8,9-tetrahydropyrido[1,2-a]indol-10-yl}-acetic acid or a pharmaceutically acceptable salt thereof.

17. The method of claim 6, wherein the leukotriene receptor antagonist is montelukast.
